(19) United States
(12) Patent Application Publication
(10) Pub. No.: US 2026/0263405 A1
(43) Pub. Date: Sep. 10, 2026

(54) Method of Treating Cancer

(71) Applicant: University of Tennessee Research Foundation, Memphis, TN (US)

(72) Inventors: Liza Makowski Hayes, Memphis, TN (US); Margaret S Bohm, Memphis, TN (US); Joseph F Pierre, Memphis, TN (US)

(73) Assignee: University of Tennessee Research Foundation, Memphis, TN (US)

(21) Appl. No.: 19/557,463

(22) Filed: Mar. 5, 2026

Related U.S. Application Data
(60) Provisional application No. 63/767,944, filed on Mar. 6, 2025.

(51) Int. Cl. A61K 31/198; A61K 31/43; A61K 31/546; A61K 31/7036; A61K 35/38; A61K 38/14; A61K 45/06; A61P 35/00

(52) U.S. Cl. CPC A61K 31/198; A61K 31/43; A61K 31/546; A61K 31/7036; A61K 35/38; A61K 38/14; A61K 45/06; A61P 35/00

(57) ABSTRACT

A method to enhance immune checkpoint blockade in breast cancer and other solid tumors and hematologic tumors by administering a PD-1/PD-L1 inhibitor together with branched-chain amino acids (leucine, isoleucine, valine) or a post-bariatric donor fecal microbiota transplantation (FMT) in order to increase circulating BCAA levels, activate iNKT1 cells within the tumor microenvironment, and improve therapeutic response.

### CROSS REFERENCE TO RELATED APPLICATION

This application claims the benefit of U.S. Provisional Patent Application Ser. No. 63/767,944, filed Mar. 6, 2025, which is incorporated herein by reference.

### STATEMENT OF FEDERALLY SPONSORED RESEARCH

This invention was made with government support under grant numbers NCI R01CA253329 and U01CA272541 awarded by the National Institutes of Health. The government has certain rights in the invention.

### TECHNICAL FIELD

The present invention relates generally to the fields of oncology, obesity, immunology, and microbiology. More specifically, the invention relates to methods and compositions for modulating the gut microbiome by bariatric surgery to enhance the efficacy of immunotherapy, particularly in the context of breast cancer treatment through regulation of microbial metabolites.

### BACKGROUND

Breast cancer (BC) is the leading cause of cancer-related death among females worldwide. Patients with a history of metabolic weight loss surgery, or bariatric surgery, have reduced risk of cancer onset, as well as improved responses to immunotherapy, lower stage of severity at diagnosis, longer time to recurrence, and decreased mortality upon subsequent BC diagnosis. However, the mechanisms behind these beneficial outcomes remain unknown.

Bariatric surgery has been increasing in prevalence throughout the past decades due to recent advances in laparoscopic approaches for obese patients. Retrospective studies demonstrate that bariatric surgery, including both vertical sleeve gastrectomy (VSG) and Roux-en-Y gastric bypass, is associated with improved BC outcomes up to 10 years post-surgery. ER-negative BC shows the most significant decrease in cancer-related mortality regardless of the amount of weight lost. VSG in a preclinical obese model has been shown to reduce BC tumor progression and improve response to immunotherapy, but the specific mechanisms behind the cancer protection after VSG remain undefined.

One potential mechanism for bariatric surgery-induced improvements in tumor progression and superior response to therapy is changes to the microbiome and its associated metabolites. Obesity is associated with dysbiosis of the gut microbiome characterized by decreased microbial diversity and increased abundance of both classically pathogenic species and SCFA-producing bacteria. Composition of the gut microbiome is altered in patients following bariatric surgery. These changes persist beyond the initial weight loss period of 12 months out to more than a decade post-surgery. Bariatric surgery in both patients and preclinical models results in an intermediate microbiome phenotype with notably increased microbial diversity, though surgery does not result in a complete return to a lean gut microbiome composition. Previous work has demonstrated that fecal microbial transplant (FMT) of microbes from mice with previous bariatric surgery into non-operated, germ-free mice induces weight loss, indicating that the gut microbiome plays a role in the beneficial impacts of bariatric surgery. Importantly, clinical studies demonstrate that the gut microbiome impacts onset, progression, and response to chemo- and immunotherapies in certain types of cancer, including extraintestinal cancers such as BC. FMTs are currently under active investigation for their potential therapeutic uses in treatment for many types of cancer. The gut microbiome potentially acts through producing or modifying microbially derived metabolites such as short chain fatty acids, branched chain amino acids (BCAA), and bile acids, which can impact anti-tumor immunity.

### SUMMARY

The present invention addresses these deficiencies by providing compositions and methods for augmenting cancer immunotherapy response through gut microbiome modulation and BCAA administration, thereby activating invariant natural killer T cells (iNKT1)-mediated anti-tumor immunity and converting immune checkpoint inhibitor-resistant tumors to responsive phenotypes in breast cancer and other solid tumors.

Bariatric surgery is associated with improved breast cancer outcomes, including greater immunotherapy effectiveness in a pre-clinical breast cancer model. A potential mechanism of bariatric surgery-associated protection is gut microbiota. The present disclosure demonstrates the dependency of improved immunotherapy response on the post-bariatric surgery gut microbiome via fecal microbial transplant (FMT). Response to αPD-1 immunotherapy in lean recipient mice with breast cancer was significantly improved following fecal microbial transplant from formerly obese bariatric surgery-treated mice. When stool samples from post-bariatric surgery patients were transplanted into recipient mice and compared to the patients' pre-surgery transplants, post-surgery microbes significantly reduced tumor burden and doubled immunotherapy effectiveness. Taken together, bariatric surgery changes the gut microbiome and transplant of that fecal sample improves response to immunotherapy.

Microbes impact tumor burden through microbially derived metabolites, including BCAAs. Circulating BCAAs correlated significantly with natural killer T (NKT) cell content in the tumor microenvironment in donor mice after bariatric surgery and fecal microbial transplant recipients of donor cecal content after bariatric surgery compared to obese controls. BCAA supplementation replicated improved αPD-1 effectiveness in two breast cancer models, supporting the role of BCAA in increased immunotherapy effectiveness after bariatric surgery. Ex vivo exposure increased primary invariant NKT cell expression of anti-tumor cytokines, demonstrating direct activation of invariant NKT cells by branched chain amino acids. Together, findings suggest that reinvigorating anti-tumor immunity may depend upon bariatric surgery-associated microbially derived metabolites, namely BCAAs.

The present invention relates to compositions and methods for augmenting immune checkpoint blockade (ICB) therapy in cancer patients through administration of BCAAs and/or gut microbiome modulation via fecal microbiota transplantation (FMT) derived from post-bariatric surgery donors. The inventors have discovered that bariatric surgery-associated restructuring of the gut microbiome enriches bacterial taxa of order Clostridiales, including family Peptostreptococcaceae, or other microbes which elevate circulating BCAA concentrations comprising leucine, isoleucine, and valine. These elevated BCAAs activate invariant natural killer T (iNKT1) cells within the tumor microenvironment, driving production of interferon-gamma (IFN-γ) and tumor necrosis factor-alpha (TNF-α) and significantly improving therapeutic response to anti-programmed cell death protein-1 (anti-PD-1) immune checkpoint inhibitors. Critically, the invention provides methods capable of converting tumors resistant to immune checkpoint inhibitor monotherapy into immunotherapy-responsive phenotypes, addressing a critical unmet need given that fewer than 25% of triple-negative breast cancer patients currently respond to ICB therapy.

The invention further provides a method combining FMT from a post-bariatric surgery donor using BCAA supplementation and immune checkpoint inhibitor administration, wherein the BCAAs increase iNKT1 cell numbers and activation, producing IFN-γ and TNF-α within the tumor microenvironment. In a particularly significant embodiment, this combination sensitizes triple-negative breast cancer tumors that are refractory to immune checkpoint inhibitor monotherapy to ICB therapy through enhanced iNKT1 cell infiltration and activation.

The invention further provides a method of augmenting ICB therapy in subjects with solid tumors, including breast cancer, triple-negative breast cancer, colorectal cancer, melanoma, lung cancer, pancreatic cancer, liver (hepatocellular), and other solid tumors and hematologic malignancies (including Chronic Lymphocytic Leukemia (CLL), Multiple Myeloma, and Acute Myeloid Leukemia (AML)) in which iNKT1 cell activation is associated with anti-tumor immune response, comprising co-administration of an anti-PD-1 or anti-PD-L1 immune checkpoint inhibitor with BCAAs sufficient to increase circulating BCAA plasma concentration by at least 10% relative to pre-treatment baseline.

The invention further provides a fecal microbiota transplantation composition for augmenting immune checkpoint blockade therapy, comprising a microbial community derived from a post-bariatric surgery donor characterized by enrichment of order Clostridiales, including family Peptostreptococcaceae, or other microbes, relative to obese non-surgical controls as determined by 16S rRNA or metagenomic shotgun sequencing, and demonstrating increased capacity for BCAA biosynthesis as determined by gas chromatography-mass spectrometry metabolomic profiling.

The invention further provides a pharmaceutical kit comprising: (a) a BCAA formulation comprising leucine, isoleucine, and valine in approximately equal amounts by weight; (b) an immune checkpoint inhibitor targeting PD-1 or PD-L1; and (c) instructions for co-administration in an amount sufficient to increase circulating BCAA plasma concentration by at least 10% relative to pre-treatment baseline, thereby activating iNKT1 cells within the tumor microenvironment and improving therapeutic response.

The present invention provides several significant advantages over existing therapy. First, BCAA supplementation is orally administrable, low-cost, and well-tolerated, requiring no modification to existing ICB regimens. Second, FMT transfers the cancer-protective microbiome benefits of bariatric surgery to non-surgical patients, expanding access beyond the surgical population. Third, and most significantly, both BCAA supplementation and FMT convert ICB-resistant tumors to ICB-responsive phenotypes through iNKT1 cell activation, directly addressing the critical unmet need in triple-negative breast cancer. Fourth, measurement of circulating BCAA plasma concentration provides a companion diagnostic biomarker enabling precision patient selection for the therapeutic methods disclosed herein. Fifth, unlike prior art methods, the present invention utilizes naturally occurring, non-recombinant BCAA-producing Clostridiales bacteria to activate iNKT1 cells through a ligand-independent metabolic priming mechanism that converts ICB-resistant solid tumors to ICB-responsive phenotypes.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present disclosure will become more readily apparent from the Description of Embodiments, particularly when taken together with the drawings.

### BRIEF DESCRIPTION OF FIGURES

The subject matter regarded as the invention is particularly pointed out and distinctly claimed in the concluding portion of the specification. The invention may be further understood by reference to the following detailed description when read with the accompanying figures.

FIG. 1A, FIG. 1B and FIG. 1C show the mouse-to-mouse fecal microbial transplant study design. FIG. 1A shows an illustration of mouse-to-mouse fecal microbial transplant following VSG. FIG. 1B shows mouse-to-mouse FMT gavages were derived from cecal contents collected at endpoint from mice who were lean and received a sham surgery (lean FMT), obese and received a sham surgery (obese FMT), weight-matched calorically restricted and received a sham surgery (WM FMT), and formerly obese who lost weight following VSG (VSG FMT). FIG. 1C shows mouse-to-mouse FMT study schema: female C57BL/6J mice were purchased from The Jackson Laboratory at 4 weeks of age and placed on LFD. On day-13, a broad-spectrum antibiotic cocktail (Neomycin 1 g/L, Ampicillin 1 g/L, Cefoperazone 1 g/L, and Vancomycin 0.5 g/L) was administered for 8 days via drinking water to ablate the commensal gut microbiome prior to fecal microbial transplant (FMT). Following 2 FMT gavages (FMT A&B), E0771 breast cancer cells were injected into the 4th right mammary fat pad and monitored for 3 weeks. Two additional FMT gavages occurred following cell injection (FMT C&D). αPD-1 immune checkpoint blockade (ICB) therapy or IgG2a isotype control was administered at 200 μg/mouse intraperitoneally every 3 days from tumor injection to endpoint. Cartoons created with BioRender.

FIG. 2A, FIG. 2B and FIG. 2C show the human-to-mouse fecal microbial transplant study design. FIG. 2A shows an illustration of mouse-to-mouse fecal microbial transplant following VSG. FIG. 2B shows FMT gavages were derived from stool collected from patients before bariatric surgery (pre-surgery FMT), or 6 months post-bariatric surgery (post-surgery FMT) transplanted into separate recipient groups. FIG. 2C shows human-to-mouse FMT study schema: female C57BL/6J mice were purchased from The Jackson Laboratory at 4 weeks of age and placed on Western or Mediterranean diets. On day-13, a broad-spectrum antibiotic cocktail (neomycin 1 g/L, ampicillin 1 g/L, cefoperazone 1 g/L, and vancomycin 0.5 g/L) was administered for 8 days via drinking water to ablate the commensal gut microbiome prior to fecal microbial transplant (FMT). Following 2 FMT gavages (FMT A&B), E0771 breast cancer cells were injected into the 4th right mammary fat pad and monitored for 3 weeks. Two additional FMT gavages occurred following cell injection (FMT C&D). αPD-1 immune checkpoint blockade (ICB) therapy or IgG2a isotype control was administered at 200 μg/mouse intraperitoneally every 3 days from tumor injection to endpoint. Cartoons created with BioRender.

FIG. 3A and FIG. 3B shows that microbes transplanted from VSG donors moderately reduced tumor burden. FIG. 3A shows that tumor progression was recorded by digital caliper with control mice (ABX−/FMT−) in the dashed black line, antibiotics only control in the dashed orange line (ABX+/FMT−), and mice receiving microbes through FMT from donors in the solid lines. Significance was determined by two-way ANOVA mixed effect model with Fisher's LSD test; N=9-10 per group. FIG. 3B shows tumor volume at endpoint presented as mean±SEM with comparisons determined by one-way ANOVA. N=10 per group. Black dashed lines correspond to ABX−/FMT− controls, orange dashed lines correspond to ABX+/FMT− controls, gray corresponds to lean FMT, black to obese FMT, blue to WM sham FMT, and red to VSG FMT.

FIG. 4A, FIG. 4B, FIG. 4C and FIG. 4D show microbes transplanted from VSG donors improved response to αPD-1 immune checkpoint blockade therapy. FIG. 4A shows tumor progression over time for FMT groups with IgG2a controls in black and αPD-1 ICB groups in gray. Significance was determined by two-way ANOVA mixed effect model with Fisher's LSD test; N=8-10 per group. FIG. 4B shows tumor volume of FMT groups at endpoint presented as mean±SEM with comparisons determined by two-way ANOVA. N=8-10 per group. FIG. 4C shows tumor progression recorded by digital caliper with control mice (ABX−/FMT) in the dashed black line, antibiotics only control in the dashed orange line (ABX+/FMT−), and mice receiving microbes through FMT from donors in the solid lines. Significance was determined by two-way ANOVA mixed effect model with Fisher's LSD test; N=9-10 per group. FIG. 4D shows tumor volume at endpoint presented as mean±SEM with comparisons determined by one-way ANOVA. N=10 per group. Black corresponds to IgG2a isotype control and gray corresponds to αPD-1 ICB treatment. *p<0.05.

FIG. 5 shows microbes from donors pre- and post-surgery do not impact body weight of recipients on Mediterranean diet. Antibiotic water intake was measured as mass in grams at the cage level over the course of an 8-day antibiotic treatment. All cages received antibiotic cocktail. N=2 cages per donor per group, with N=5 mice per cage. Body weights were measured over the duration of the study. Significance was determined by two-way ANOVA mixed effects model with Fisher's LSD test; N=10 per donor per group. Data are presented as mean±SEM with comparisons determined by two-way ANOVA. N=10 per donor per group. Black corresponds to IgG2a isotype control and gray corresponds to αPD-1 ICB treatment. *p<0.05.

FIG. 6 shows microbes from donors pre- and post-surgery do not impact body weight of recipients on Western diet. Antibiotic (ABX) water intake was measured as mass in grams at the cage level over the course of an 8-day ABX treatment. All cages received ABX cocktail. N=2 cages per donor per group, with N=5 mice per cage. Body weights were measured over the duration of the study. Significance was determined by two-way ANOVA mixed effects model with Fisher's LSD test; N=10 per donor per group. Data are presented as mean±SEM with comparisons determined by two-way ANOVA. N=10 per donor per group. Black corresponds to IgG2a isotype control and gray corresponds to αPD-1 ICB treatment.

FIG. 7A, FIG. 7B, FIG. 7C, FIG. 7D, FIG. 7E and FIG. 7F show microbes from patients post-bariatric surgery reduced tumor burden and improved response to ICB in recipient mice on Mediterranean diet. FIG. 7A shows human to mouse fecal microbial transplant-lean recipient mice fed a Mediterranean diet tumor progression following FMT from Donor A was recorded by digital caliper. N=8-10 recipients per donor group. FIG. 7B shows tumor volume at endpoint following FMT from Donor A presented as mean±SEM with comparisons determined by two-way ANOVA. FIG. 7C shows tumor regression following FMT from Donor A presented as a percent change in tumor volume is shown as rank-sorted treatment groups. FIG. 7D shows human to mouse fecal microbial transplant-lean recipient mice fed a Mediterranean diet tumor progression following FMT from Donor B was recorded by digital caliper. N=10 recipients per donor group. FIG. 7E shows tumor volume at endpoint following FMT from Donor B, presented as mean±SEM with comparisons determined by two-way ANOVA. FIG. 7F shows tumor regression following FMT from Donor B, presented as percent change in tumor volume is shown as rank-sorted treatment groups. Black corresponds to IgG2a isotype control and gray corresponds to αPD-1 ICB treatment. *p<0.05, ***p<0.001, ****p<0.0001.

FIG. 8A, FIG. 8B and FIG. 8C show microbes from patients post-bariatric surgery reduced tumor burden and improved response to ICB in recipient mice on a Western diet. FIG. 8A shows human to mouse fecal microbial transplant-lean recipient mice fed a Western pattern diet tumor progression following FMT from Donor A was recorded by digital caliper. N=10 recipients per donor group. FIG. 8B shows tumor volume at endpoint following FMT from Donor A presented as mean±SEM with comparisons determined by two-way ANOVA. FIG. 8C shows tumor regression following FMT from Donor A presented as percent change in tumor volume is shown as rank-sorted treatment groups. Black corresponds to IgG2a isotype control and gray corresponds to αPD-1 ICB treatment. *p<0.05, ***p<0.001, ****p<0.0001.

FIG. 9A, FIG. 9B, FIG. 9C, FIG. 9D, FIG. 9E and FIG. 9F show microbes transplanted from VSG donors demonstrate elevated Order Clostridiales in response to αPD-1 immune checkpoint blockade therapy. FIG. 9A shows relative abundance of microbial families reported from donor cecal contents harvested from obese sham or VSG donors at endpoint. Relative abundance was calculated using top 1000 taxa identified using phyloseq. N=6-7 per group. FIG. 9B shows alpha diversity in recipients across the timeline of the study included baseline stool collected at Day-13, post-antibiotic stool collected at Day-5, and cecal contents taken at endpoint. Shannon and Simpson indices were calculated using phyloseq. N=8-10 per group for VSG FMT recipients. FIG. 9C shows relative abundance of microbial families is reported in recipient cecal contents harvested at FMT study endpoint. Relative abundance was calculated using top 1000 taxa identified using phyloseq. N=8-10 per group. FIG. 9D and FIG. 9E show differential abundance analysis of endpoint cecal contents for family Peptostreptococcaceae (FIG. 9D) and Order Clostridiales (FIG. 9E). Significant comparisons were determined by two-way ANOVA of relative abundance values. N=9-10 per group. Black corresponds to IgG2a isotype control and gray corresponds to αPD-1 ICB treatment. *p<0.05. FIG. 9F shows abundance of Order Clostridiales members in the cecal contents at endpoint were correlated with endpoint tumor volume via MaAsLin2 analysis. R²=0.12, p=0.037, N=38.

FIG. 10A, FIG. 10B, FIG. 10C and FIG. 10D show elevated circulating branched chain amino acids were detected after FMT of VSG donor microbes. FIG. 10A, FIG. 10B and FIG. 10C show circulating individual BCAAs correlated with tumor volume at endpoint via linear regression analysis for valine (FIG. 10A, R²=0.12, p=0.037, N=36), leucine (FIG. 10B, R²=0.14, p=0.022, N=36), and isoleucine (FIG. 10C, R²=0.13, p=0.034, N=36). FIG. 10D shows the abundance of Order Clostridiales members in cecal contents at endpoint were correlated with relative concentration of circulating branched chain amino acids via MaAsLin2 analysis. R²=0.28, p=0.029, N=17.

FIG. 11A, FIG. 11B and FIG. 11C show elevated circulating branched chain amino acids correlated with improved anti-tumor immune response and NKT cells. FIG. 11A shows flow cytometric analysis of the tumor immune microenvironment (TIME) as frequency of NK/NKT cells (NK1.1+) out of total CD45+ immune cells. Data are presented as mean±SEM with two-way ANOVA comparisons for N=6-9 per group. Black corresponds to IgG2a isotype control and gray corresponds to αPD-1 ICB treatment. ***p<0.001, ****p<0.0001. FIG. 11B shows the percentage of NK/NKT cells out of total immune cells within the TIME were correlated with tumor volume at endpoint via linear regression analysis. R²=0.27, p=0.002, N=33. FIG. 11C shows the percentage of NK/NK T cells out of total immune cells within the TIME were correlated with circulating BCAA levels via linear regression analysis. R²=0.16, p=0.034, N=29.

FIG. 12A and FIG. 12B show that in donor cecal content, vertical sleeve gastrectomy modulated the microbially derived metabolite profile and elevated intra-tumoral NKT cells. FIG. 12A shows NKT cell enrichment within the TIME of FMT donors calculated using xCell algorithm in Timer2.0 on bulk RNAseq data to identify immune population signatures. Significant comparisons determined by Student's t-test; N=7-8. Black corresponds to obese sham group and gray corresponds to VSG group. *p<0.05. FIG. 12B shows circulating BCAA concentrations from FMT donors was correlated with NK T cell enrichment scores within the TIME via linear regression analysis. R²=0.46, p=0.011, N=11.

FIG. 13A, FIG. 13B, FIG. 13C, FIG. 13D, FIG. 13E, FIG. 13F, and FIG. 13G show that branched chain amino acid supplementation mimics the effects of the post-bariatric surgery microbiome. FIG. 13A shows the branched chain amino acid (BCAA) supplementation study schema: female C57BL/6J or BALB/cJ mice were purchased from The Jackson Laboratory at 4 weeks of age and placed on LFD. On day-7, a BCAA cocktail (leucine 15 g/L, isoleucine 15 g/L, and valine 15 g/L) was administered via drinking water prior to breast cancer cell injections. E0771 or 4T1 BC cells were injected into the 4th right mammary fat pad of C57BL/6J or BALB/cJ mice and monitored for 2-3 weeks. αPD-1 immune checkpoint blockade (ICB) therapy or IgG2a isotype control was administered at 200 μg/mouse intraperitoneally every 3 days from tumor injection to endpoint. FIG. 13B shows BCAA water intake measured as mass in grams at the cage level over the course of the entire 22-day supplementation. Vehicle cages received regular autoclaved drinking water. N=2 cages per group, with N=5 mice per cage. FIG. 13C shows body weights measured over the duration of the study. Significance was determined by two-way ANOVA mixed effects model with Fisher's LSD test; N=10 per group. FIG. 13D shows E0771 tumor progression in C57BL/6J mice recorded by digital caliper. N=9-10 per group. FIG. 13E shows BCAA water intake measured as mass in grams at the cage level over the course of the entire 20-day supplementation. Vehicle cages received regular autoclaved drinking water. N=1 cage per group, with N=5 mice per cage. FIG. 13F shows body weights measured over the duration of the study. Significance was determined by two-way ANOVA mixed effects model with Fisher's LSD test; N=5 per group. FIG. 13G shows 4T1 tumor progression in BALB/cJ mice recorded by digital caliper. N=5 per group. Cartoon created with BioRender.

FIG. 14A, FIG. 14B, FIG. 14C, FIG. 14D, FIG. 14E, FIG. 14F, and FIG. 14G show branched chain amino acid supplementation increases iNKT cell content in BC tumors. FIG. 14A shows flow cytometric analysis of invariant NKT (iNKT) cells (CD3+, PBS57-loaded mouse CD1d tetramer+) out of total CD3+ cells. Mean±SEM with two-way ANOVA for N=5-9 per group. FIG. 14B shows the percentage of iNKT cells out of total CD3+ cells within E0771 TIME were correlated with tumor volume at endpoint via linear regression analysis. R²=0.13, p=0.046, N=30. FIG. 14C shows flow cytometric analysis of 4T1 TIME shown as frequency of iNKT cells (PBS57-loaded mouse CD1d tetramer+) out of total CD45+ cells. Data presented as mean±SEM with two-way ANOVA for N=5 per group. FIG. 14D shows the percentage of iNKT cells out of total CD45+ cells within 4T1 TIME were correlated with tumor volume at endpoint via linear regression analysis. R²=0.45, p=0.001, N=20. FIG. 14E, FIG. 14F and FIG. 14G show CD69+ iNKT cells (FIG. 14D), IFNγ+ iNKT cells (FIG. 14F), and TNFα+ iNKT cells (FIG. 14G) out of total iNKT cells. Data presented as mean±SEM with two-way ANOVA for N=5 per group. Black corresponds to IgG2a isotype control and gray corresponds to αPD-1 ICB treatment. *p<0.05, ***p<0.001, ****p<0.0001.

FIG. 15A, FIG. 15B and FIG. 15C show post-surgery FMT from Donor B increases iNKT cells in the TIME. FIG. 15A and FIG. 15B show flow cytometry analysis of immune cell populations in tumor immune microenvironment (TIME) from human-to-mouse FMT Donor B in FIG. 4-10 is shown as frequency of iNKT cells out of total CD45+ cells (FIG. 15A) and frequency of CD69+ iNKT cells out of total iNKT cells (FIG. 15B). Data are presented as mean±SEM with two-way ANOVA analyses; N=10 per group. FIG. 15C shows the percentage of iNKT cells out of total CD45+ cells within TIME were correlated with tumor volume at endpoint via linear regression analysis. R²=0.22, p=0.002, N=40. Data are presented as mean±SEM with two-way ANOVA analyses; N=10 per group. Black corresponds to IgG2a isotype control and gray corresponds to αPD-1 ICB treatment. ***p<0.001, ****p<0.0001.

FIG. 16A, FIG. 16B, FIG. 16C, FIG. 16D and FIG. 16E show that branched chain amino acid treatment increases iNKT cell production of proinflammatory cytokines. FIG. 16A shows the study schema: liver lymphocytes were isolated from female C57BL/6J mice and fluorescence-activated cell sorting (FACS) was used to isolate iNKT cells via PBS57-loaded mouse tetramer staining. Cells were expanded in culture for 20 days. On day 20, cells were treated with a branched chain amino acid (BCAA) cocktail (leucine 10 mM, isoleucine 10 mM, and valine 10 mM) or vehicle control for 24 hours. On Day 21, cells were stained for flow cytometry analysis. FIG. 16B, FIG. 16C and FIG. 16D show flow cytometric analysis of cultured cells at endpoint shown as iNKT cells (FIG. 16B, PBS57-loaded mouse tetramer+) out of total live cells, IFNγ+ cells (FIG. 16C) and TNFα+ cells (FIG. 16D) out of iNKT cells. Data presented as mean±SEM with Student's t-test for N=4 wells per group. Black corresponds to vehicle control and gray corresponds to BCAA treatment. *p<0.05, **p<0.01. FIG. 16E shows a proposed model: NKT cells are induced to mature by dendritic cells. Mature NKT cells activate anti-tumor immune cells including T cells and NK cells which induce cancer cell death. Findings presented suggest that BCAAs are elevated in mice after FMT from VSG donors and are essential drivers of the NKT cell anti-tumor immune response. Solid lines indicate known interactions while dashed lines represent proposed interactions. Cartoon created with BioRender.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present disclosure generally relates to methods for improving therapeutic response to immune checkpoint blockade in subjects having breast cancer, other solid tumors, or hematologic malignancies by modulating metabolism and anti-tumor immunity. In various embodiments, the methods comprise administering a PD-1 and/or PD-L1 immune checkpoint inhibitor together with BCAAs including leucine, isoleucine, and valine in an amount sufficient to increase circulating BCAA plasma concentration (e.g., by at least 10% relative to a pre-treatment baseline), thereby activating invariant natural killer T (iNKT) cells within the tumor microenvironment, as evidenced by increased iNKT1 frequency/infiltration, activation marker (e.g., CD69), and/or production of effector cytokines such as IFN-γ and TNF-α. In certain implementations, BCAAs are orally administered, optionally at weight-based doses and ratios, initiated prior to checkpoint therapy, and titrated based on measured circulating BCAA levels and/or iNKT activation, with dose reduction or discontinuation if activation exceeds a predefined threshold. Additional embodiments employ fecal microbiota transplantation from post-bariatric surgery donors selected based on microbiome and/or metabolomic profiles (e.g., enrichment of Clostridiales including Peptostreptococcaceae or other microbes and elevated microbially derived BCAAs), optionally following antibiotic-mediated microbiome ablation, to increase circulating BCAAs and enhance iNKT activation, alone or in combination with BCAA supplementation, including in checkpoint-resistant or refractory settings such as triple-negative breast cancer, with therapeutic benefit assessed by tumor burden reduction and/or conversion from checkpoint-nonresponsive to checkpoint-responsive phenotypes. In certain embodiments, BCAA may be used to activate chimeric antigen receptor (CAR)-iNKT cells.

In some embodiments, a method of treating breast cancer in a subject in need thereof comprises administering to the subject an immune checkpoint inhibitor that blocks programmed cell death protein-1 (PD-1) or programmed death-ligand 1 (PD-L1), and administering to the subject an effective amount of BCAAs comprising leucine, isoleucine, and valine. In certain embodiments, the BCAAs are administered in a total daily dose sufficient to increase circulating BCAA plasma concentration by at least 10% relative to a pre-treatment baseline. In some embodiments, the BCAA administration activates invariant natural killer T (iNKT) cells within the tumor microenvironment, as determined by an increase in iNKT1 cell frequency and/or an increase in activation marker expression relative to pre-treatment baseline, thereby improving therapeutic response to the immune checkpoint inhibitor.

In another embodiment, the BCAAs are administered orally. In some embodiments, the oral BCAAs are provided as a nutritional supplement, a pharmaceutical composition, and/or an enteral formulation suitable for delivery to the gastrointestinal tract, such that the subject receives leucine, isoleucine, and valine in effective amounts.

In another embodiment, the total daily BCAA dose is from about 0.1 g/kg/day to about 1.0 g/kg/day. In certain embodiments, the daily dose is selected and/or titrated based on achieving a target increase in circulating BCAA plasma concentration of at least 10% relative to pre-treatment baseline, as measured using a quantitative assay.

In a further embodiment, leucine, isoleucine, and valine are administered in approximately equal amounts by weight. In some embodiments, the BCAAs are provided at a leucine:isoleucine:valine ratio of about 1:1:1. In certain embodiments, BCAA administration is initiated at least seven days prior to initiation of immune checkpoint inhibitor therapy and continued for the duration of treatment with the immune checkpoint inhibitor.

In another embodiment, iNKT1 cells activated within the tumor microenvironment are characterized by production of interferon-gamma (IFN-γ) and/or tumor necrosis factor-alpha (TNF-α). In some embodiments, such cytokine production is determined by intracellular cytokine detection, flow cytometry, ELISPOT, immunohistochemistry, or another equivalent immunological assay performed on tumor-derived cells and/or tumor-associated immune infiltrates.

In another embodiment, the BCAAs are administered in combination with the immune checkpoint inhibitor to a subject whose breast cancer is resistant or refractory to immune checkpoint inhibitor monotherapy. In some embodiments, the BCAA administration sensitizes the tumor to immune checkpoint inhibitor therapy by increasing iNKT1 cell infiltration within the tumor microenvironment relative to pre-treatment baseline, thereby improving response in a subject who previously exhibited insufficient benefit from checkpoint blockade alone.

In another embodiment, the subject is a human patient having triple-negative breast cancer. In certain embodiments, the patient has failed immune checkpoint inhibitor monotherapy or is refractory to immune checkpoint inhibitor monotherapy prior to initiation of the combination approach that includes BCAA administration.

In further embodiment comprises measuring iNKT cell activation in the tumor microenvironment and/or peripheral blood of the subject, and adjusting the BCAA dose based on the measurement. In some embodiments, iNKT activation is assessed by one or more of: (i) measuring iNKT cell frequency using CD1d tetramer staining or an equivalent iNKT cell-specific detection reagent; (ii) measuring expression of a CD69 activation marker; and/or (iii) measuring intracellular production of IFN-γ and/or TNF-α. In certain embodiments, the BCAA dose is increased, maintained, or decreased to achieve a desired increase in iNKT activation relative to pre-treatment baseline.

Additional embodiments include discontinuing or reducing BCAA administration when iNKT cell activation exceeds a predefined upper threshold. In some embodiments, the upper threshold is selected as being indicative of potential immune-related adverse events, and the BCAA dosing is modified to reduce activation to within a predetermined target range while continuing immune checkpoint inhibitor therapy as clinically appropriate.

Additional embodiments include measuring circulating BCAA plasma concentration in the subject prior to treatment initiation by a quantitative analytical method. In some embodiments, a circulating BCAA plasma concentration below a predefined threshold identifies the subject as a candidate for BCAA supplementation prior to or concurrent with administration of the immune checkpoint inhibitor, thereby supporting patient selection and/or dosing decisions.

In another embodiment, a method of treating breast cancer in a subject in need thereof is provided, which comprises administering to the subject an immune checkpoint inhibitor that blocks PD-1 or PD-L1, and administering to the subject a fecal microbiota transplantation (FMT) composition obtained from a post-bariatric surgery human donor. In certain embodiments, the donor has undergone bariatric surgery at least three months prior to donation and achieved at least a 10% reduction in body weight relative to pre-surgical weight. In some embodiments, the FMT composition is characterized by enrichment of order Clostridiales, including family Peptostreptococcaceae, or other microbes relative to obese non-surgical controls. In certain embodiments, administration of the FMT composition increases circulating BCAA levels and iNKT cell activation within the tumor microenvironment of the subject relative to pre-treatment baseline, thereby improving the subject's therapeutic response to the immune checkpoint inhibitor.

In a further embodiment the subject undergoes gut microbiome ablation using a broad-spectrum antibiotic regimen prior to administration of the FMT composition. In some embodiments, the antibiotic regimen comprises one or more of vancomycin, neomycin, ampicillin, and cefoperazone (or an equivalent broad-spectrum antibiotic regimen) administered for at least five days prior to FMT. In certain embodiments, the FMT composition is administered in at least two doses at intervals of no more than three days apart prior to initiation of immune checkpoint inhibitor therapy.

In a further embodiment the post-bariatric surgery human donor is selected based on a fecal microbiome profile demonstrating enrichment of order Clostridiales, including family Peptostreptococcaceae, or other microbes relative to obese non-surgical controls. In some embodiments, the fecal microbiome profile is determined by a nucleic acid-based microbial profiling method, including 16S rRNA gene sequencing, metagenomic sequencing, qPCR-based assays, or an equivalent profiling technique.

In a further embodiment the post-bariatric surgery human donor is selected based on stool metabolomic profiling demonstrating microbially derived BCAA levels above a predefined threshold relative to obese non-surgical controls. In some embodiments, stool metabolomic profiling is performed using a quantitative analytical method comprising mass spectrometry, liquid chromatography-mass spectrometry, nuclear magnetic resonance spectroscopy, or an equivalent quantitative platform.

In a further embodiment the improvement in therapeutic response comprises one or more of: (i) reduction in tumor volume of at least 50% relative to untreated controls; (ii) tumor regression to an impalpable state; and/or (iii) conversion of an immune checkpoint inhibitor-resistant tumor phenotype to an immune checkpoint inhibitor-responsive phenotype. In some embodiments, tumor burden is measured by caliper assessment, an imaging modality, and/or an equivalent tumor burden measurement technique.

In some embodiments, a method of treating breast cancer in a subject in need thereof comprises administering to the subject an immune checkpoint inhibitor that blocks PD-1 or PD-L1; administering to the subject an FMT composition obtained from a post-bariatric surgery human donor who has undergone bariatric surgery at least three months prior to donation and achieved at least a 10% reduction in body weight relative to pre-surgical weight; and administering to the subject an effective amount of BCAAs comprising leucine, isoleucine, and valine in a total daily dose sufficient to increase circulating BCAA plasma concentration by at least 10% relative to pre-treatment baseline. In certain embodiments, the FMT composition and the BCAAs together increase circulating BCAA levels and activate iNKT1 cells within the tumor microenvironment, wherein the activated iNKT1 cells produce IFN-γ and/or TNF-α, thereby improving a therapeutic response of the subject to the immune checkpoint inhibitor.

In a further embodiment the subject is a human patient with triple-negative breast cancer who has failed or is refractory to immune checkpoint inhibitor monotherapy. In some embodiments, combined administration of the FMT composition and BCAAs sensitizes the tumor to immune checkpoint inhibitor therapy by increasing iNKT1 cell infiltration and activation within the tumor microenvironment relative to pre-treatment baseline.

In another embodiment provided herein is a method of augmenting immune checkpoint blockade therapy in a subject with a solid tumor comprises administering to the subject an immune checkpoint inhibitor that blocks PD-1 or PD-L1, and administering to the subject an effective amount of BCAAs comprising leucine, isoleucine, and valine sufficient to increase circulating BCAA plasma concentration by at least 10% relative to pre-treatment baseline. In certain embodiments, the BCAA administration activates iNKT1 cells within the tumor microenvironment and improves a therapeutic response to the immune checkpoint inhibitor. In some embodiments, the solid tumor is breast cancer, triple-negative breast cancer, colorectal cancer, melanoma, lung cancer, pancreatic cancer, liver (hepatocellular), other solid tumors and hematologic malignancies (including Chronic Lymphocytic Leukemia (CLL), Multiple Myeloma, and Acute Myeloid Leukemia (AML)) in which iNKT1 cell activation within the tumor microenvironment is associated with an anti-tumor immune response.

The invention disclosed herein encompasses various embodiments wherein the subject of treatment may be a mammal, including humans. The therapeutic compounds and methods described herein are applicable to both non-human mammals and humans, with the aim of providing effective treatment for a wide range of medical conditions.

For non-human mammalian embodiments, the therapeutic compounds and methods may be employed in veterinary medicine for the treatment of animals such as dogs, cats, horses, cows, pigs, rodents, and other domesticated or wild mammals. These embodiments may involve administering the therapeutic compounds via suitable routes and dosage regimens tailored to the species, size, and health condition of the animal.

In certain embodiments, the methods described herein comprise administering to a subject in need thereof a programmed death-ligand 1 (PD-L1) immune checkpoint inhibitor in an amount and on a dosing schedule effective to reduce PD-L1-mediated suppression of anti-tumor immune responses and thereby enhance T-cell activity within the tumor microenvironment. The PD-L1 immune checkpoint inhibitor may be provided as a monoclonal antibody (or antigen-binding fragment or other biologic format) that specifically binds PD-L1 and blocks interaction of PD-L1 with its cognate receptors (e.g., PD-1 and/or CD80), and may be administered by any suitable route, including intravenous infusion, subcutaneous injection, or other clinically acceptable parenteral route. Administration may be performed as a single dose or as multiple doses over time (e.g., at regular intervals in a treatment cycle), and may be used alone or in combination with one or more additional therapeutic agents (e.g., chemotherapy, targeted therapy, radiotherapy, or other immunotherapies), as permitted by the intended treatment regimen. Non-limiting examples of such PD-L1 inhibitors include atezolizumab, durvalumab, and avelumab, each of which is an anti-PD-L1 monoclonal antibody that has been developed for administration in oncology indications. Additional anti-PD-L1 agents (including biosimilars, follow-on biologics, and other antibody formats such as antigen-binding fragments or engineered PD-L1-binding proteins) may likewise be employed, provided they retain the functional ability to specifically bind PD-L1 and interfere with PD-L1 signaling in a manner that enhances or restores anti-tumor immune activity. In certain embodiments, the dosage and dosing schedule of the PD-L1 immune checkpoint inhibitor may be selected based on available published literature, product labeling and/or clinical guidelines, or may be determined through the clinical judgment of the treating physician based on patient-specific factors.

Fecal matter may be collected from a human subject following bariatric surgery using conventional techniques and commercially available collection kits. In one exemplary approach, the subject is provided a sterile, single-use stool collection receptacle (e.g., a commode-mounted collection “hat” or disposable liner) configured to prevent contact with toilet water and to minimize contamination. After defecation, a designated portion of the sample is transferred, using a sterile scoop or spatula integrated with the collection container lid, into one or more pre-labeled specimen containers (e.g., leak-resistant polypropylene tubes or jars). The containers may optionally include a stabilizing medium (for example, a nucleic-acid stabilizer or preservative buffer) depending on downstream analyses. The subject (or clinical personnel) seals the container, records the date and time of collection, and places the specimen in secondary containment (e.g., a sealed biohazard bag) for transport.

Following collection, the specimen may be handled in accordance with standard clinical and laboratory practices, including short-term refrigeration (e.g., about 2-8° C.) and/or freezing (e.g., about −20° C. to −80° C.) to reduce degradation and preserve analytes prior to processing. Chain-of-custody, labeling, and de-identification procedures may be used as appropriate for clinical studies. Where post-bariatric subjects have altered stool consistency or frequency, collection instructions may include guidance for multiple time-point sampling or pooling over a defined interval, while maintaining sterile technique and avoiding cross-contamination, to obtain a representative specimen for subsequent processing, storage, and analysis.

Collected fecal matter or plasma may be analyzed for branched chain amino acids, including leucine, isoleucine, and valine, using established metabolomics workflows. In one exemplary method, an aliquot of the specimen is thawed (if frozen) under controlled conditions, homogenized in a defined mass-to-volume ratio with a cold extraction solvent (e.g., aqueous methanol or acetonitrile-containing buffer), and clarified by centrifugation to remove particulates. The supernatant may be further cleaned (e.g., filtration and/or solid-phase extraction) and spiked with isotopically labeled internal standards for leucine, isoleucine, and valine to support accurate quantification and correction for matrix effects. The resulting extract may be analyzed by liquid chromatography coupled to tandem mass spectrometry (LC-MS/MS) using either targeted multiple-reaction monitoring or high-resolution accurate-mass detection, with chromatographic conditions selected to resolve the isobaric amino acids leucine and isoleucine. Concentrations of each BCAA may be determined by comparison to external calibration curves generated from authenticated standards and reported on a normalized basis (e.g., per gram wet weight or dry weight of stool), optionally alongside quality control samples and replicate analyses to confirm precision and method performance.

In certain embodiments, collected fecal matter obtained from a human donor or autologous subject is processed under controlled conditions using aseptic technique in accordance with applicable biosafety procedures and quality-system requirements to produce an orally administrable microbiota-containing composition. The composition may be conditioned to a predetermined solids content and may be combined with pharmaceutically acceptable components selected to support stability and gastrointestinal delivery, including one or more of diluents, suspending agents, buffering agents, cryo- and/or lyoprotectants, antioxidants, moisture scavengers, and/or release-modifying materials. In exemplary implementations, the resulting material is apportioned into unit-dose oral dosage forms, including capsules, wherein the capsule shell may be selected to provide immediate release, delayed release, and/or enteric protection so as to reduce exposure to gastric conditions and promote release in the intestine. The unit doses may be placed into tamper-evident primary packaging with appropriate identification and traceability indicia and maintained under storage conditions suitable for biologic materials until administration in a supervised clinical context.

In certain embodiments, the dose and dosing regimen of the fecal matter composition administered to a subject in need thereof may be selected based on the information provided in the present disclosure as well as the available literature and/or established clinical protocols, and may be further adjusted according to the treating physician's clinical judgment in view of patient-specific factors (e.g., indication, disease severity, tolerability, and response).

Unless otherwise defined herein, scientific and technical terms used in this application shall have meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art. Unless specified otherwise, the following definitions are provided for specific terms, which are used in the above written description.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

In the context of the present invention, the term “patient” or “subject” refers to an individual, whether human or non-human mammal, who is receiving or undergoing treatment with the therapeutic compounds or methods disclosed herein. In human embodiments, the patient may be an individual diagnosed with a medical condition or disease for which treatment is indicated. In non-human mammalian embodiments, the subject may be an animal, such as a dog, cat, horse, rodent, or other mammalian species, for which therapeutic intervention is necessary or desirable. The terms “patient” or “subject” are used interchangeably throughout this specification to refer to the recipient of treatment, regardless of whether they are human or non-human mammals.

“Administration of” or “administering” refers to the act of delivering or applying the therapeutic compound to a patient or subject for the purpose of treating a medical condition or disease. Administration may be performed by healthcare professionals, caregivers, or the patients themselves, whether solely or under the guidance and supervision of qualified personnel.

Treating or treatment refers to the administration of a therapeutic agent to a patient with the intention of alleviating, curing, or preventing a disease or medical condition. The term may refer to any mode of therapy, including pharmacological, surgical, radiation, or other types of therapy. The term “preventing” may also encompass prophylactic treatment, which is the administration of a therapeutic agent to prevent the onset or recurrence of a disease or medical condition.

A “pharmaceutical composition” refers to a formulation that comprises one or more active ingredients and one or more pharmaceutically acceptable carriers. The pharmaceutical composition facilitates administration of the compound to a patient or subject. The composition may be in any suitable form, including but not limited to tablets, capsules, powders, solutions, suspensions, emulsions, gels, creams, ointments, patches, and injectable formulations. The choice of a pharmaceutical composition may depend on various factors, including the type and route of administration, the stability of the active ingredient, and the desired therapeutic effect. The composition may be prepared by any suitable method, including but not limited to blending, mixing, granulating, compressing, or lyophilizing.

A “pharmaceutically acceptable carrier” refers to a substance or a combination of substances that are inert, non-toxic, and compatible with the active ingredient(s) of the compound of the present disclosure. The carrier may be a solid, liquid, or a gas and may include, but is not limited to, excipients, diluents, binders, lubricants, disintegrants, fillers, and solvents. The choice of a pharmaceutically acceptable carrier may depend on various factors, including the type and route of administration, the stability of the active ingredient, and the desired therapeutic effect.

A “therapeutically effective amount” refers to a quantity of the active ingredient(s) of the compound of this disclosure that exhibits the desired therapeutic effect in a patient. The amount may vary depending on various factors, including the disease or disorder to be treated, the patient's age and health condition, the route of administration, and the desired therapeutic effect. A therapeutically effective amount may be determined by one skilled in the art using routine experimentation, and may be expressed as a range or a specific value.

An “Immune Checkpoint Inhibitor” or “ICI” or “ICB” refers to therapeutic agents that block inhibitory checkpoint molecules that otherwise permit cancer cells to evade immune surveillance. This includes, without limitation, antibodies targeting Programmed Cell Death Protein-1 (PD-1), Programmed Death-Ligand 1 (PD-L1), and Cytotoxic T-Lymphocyte-Associated Protein 4 (CTLA-4). Specific non-limiting examples include nivolumab, pembrolizumab, atezolizumab, and biosimilars thereof. As used herein, “immune checkpoint inhibitor that blocks PD-1 or PD-L1” encompasses all antibodies, antibody fragments, and small molecule inhibitors that disrupt PD-1/PD-L1 signaling.

A “Tumor Immune Microenvironment” or “TIME” refers to the cellular and molecular composition of the tumor and its immediate surrounding tissue, including infiltrating immune cells, stromal cells, extracellular matrix components, and soluble mediators, including cytokines and metabolites.

The compositions of the present invention can be administered through various routes to achieve therapeutic effects tailored achieve the desired therapeutic effect. These routes include oral, parenteral, rectal, topical, nasal, buccal, vaginal, and inhalation administration. Orally administered pharmaceutical compositions are ingested through the mouth and are typically in the form of tablets, capsules, solutions, or suspensions, offering convenient and widely accepted dosing options. Dosage forms and strengths can vary depending on factors such as patient age, severity of inflammation, and desired therapeutic outcome. Parenteral administration involves delivering the pharmaceutical composition directly into the body through means other than the digestive tract, such as subcutaneous (SC), intravenous (IV), intramuscular (IM), intraperitoneal (IP), or intrathecal routes. This route bypasses the gastrointestinal system, allowing for rapid absorption and systemic distribution of the therapeutic agent making it suitable for acute conditions or situations requiring immediate therapeutic intervention. Pharmaceutical compositions for intravenous administration are typically in the form of sterile solutions or suspensions. Rectal administration involves the insertion of suppositories, enemas or rectal gels into the rectum for localized or systemic effects. This route of administration is particularly useful for patients unable to take oral medications or requiring local treatment of conditions such as hemorrhoids or inflammatory bowel disease. Topical administration entails applying creams, ointments, gels, or patches directly onto the skin or mucous membranes for targeted localized treatment while minimizing systemic side effects. Nasal administration involves delivering sprays or drops into the nasal cavity for systemic or local effects. Buccal administration involves placing tablets or patches between the cheek and gum for absorption through the buccal mucosa. Vaginal administration involves the insertion of suppositories, creams, or tablets into the vagina for local or systemic effects. Inhalation administration involves inhaling aerosols, powders, or vapors into the respiratory tract for rapid absorption and distribution of the therapeutic agent. Each route of administration offers unique advantages in terms of efficacy, convenience, and patient compliance, allowing for tailored treatment approaches to optimize therapeutic outcomes.

Dosage and administration regimens may vary depending on factors such as the patient's age, weight, medical condition, and response to treatment. The dosage range may be from about 10 mg to about 1000 mg per day, depending on the disease or disorder to be treated, the patient's age and health condition, and the desired therapeutic effect. The appropriate dosage and administration schedule should be determined by a qualified healthcare professional based on individual patient characteristics and therapeutic goals.

### EXAMPLES

The following Examples are presented in order to more fully illustrate the preferred embodiments of the invention. They should in no way, however, be construed as limiting the broad scope of the invention. The following methods were used to conduct the experiments described in Examples 1-8, below:

### Reagents

All reagents were obtained from Sigma-Aldrich unless otherwise noted. Fetal bovine serum (FBS, Gibco), 100× GlutaMAX (Gibco), and 100× Penicillin/Streptomycin (HyClone) were purchased from ThermoFisher. RPMI 1640 and Matrigel are from Corning. Rodent diets were obtained from Research Diets Inc. With the exception of vancomycin (V2002 Sigma-Aldrich), antibiotics were purchased from Research Products International including neomycin (N20040), ampicillin (A40040), and cefoperazone (C57600). In vivo anti-mouse PD-1 monoclonal antibody (clone RMP1-14, BE0146) and control immunoglobulin (rat IgG2a, clone 2A3, BE0089) were obtained from BioxCell. Flow cytometry antibodies (Table 1), compensation beads, and reagents are from Tonbo Biosciences, Inc. and Biolegend. BCAAs were purchased from Sigma-Aldrich, including leucine (L8000), isoleucine (12752), and valine (V0513).

TABLE 1
Antibodies for flow cytometric analysis of tumor immune microenvironment

| Antibody | Source | Identifiers | Dilutions |
|---|---|---|---|
| Anti-Human/Mouse CD11b redFluor | Tonbo | 80-0112-U025 | 1:20 |
| 710 | | | |
| APC anti-mouse Ly-6C | BioLegend | 128015 | 1:40 |
| Anti-Mouse F4/80 Antigen PE | Tonbo | 50-4801-U025 | 1:40 |
| Anti-Mouse Ly-6G PerCP-Cyanine5.5 | Tonbo | 65-1276-U025 | 1:40 |
| Brilliant Violet 650 anti-mouse CD11c | BioLegend | 117339 | 1:40 |
| PE/Cyanine7 anti-mouse I-A/I-E | BioLegend | 107630 | 1:40 |
| (MHC class II) | | | |
| FITC anti-mouse CD45 | BioLegend | 103108 | 1:40 |
| Brilliant Violet 711 anti-mouse CD274 | BioLegend | 124319 | 1:10 |
| (B7- H1, PD-L1) | | | |
| APC/Cyanine7 anti-mouse CD103 | BioLegend | 121432 | 1:5 |
| Anti-Mouse CD8a FITC | Tonbo | 35-0081-U025 | 1:10 |
| Brilliant Violet 605 anti-mouse CD4 | BioLegend | 100548 | 1:10 |
| Brilliant Violet 785 anti-mouse CD3ε | BioLegend | 100355 | 1:4 |
| PE/Dazzle 594 anti-mouse NK-1.1 | BioLegend | 108747 | 1:10 |
| Brilliant Violet 421 anti-mouse CD279 | BioLegend | 109121 | 1:10 |
| (PD-1) | | | |
| PerCP/Cyanine5.5 anti-mouse CD45 | BioLegend | 103132 | 1:20 |
| PE anti-mouse/human CD44 | BioLegend | 103008 | 1:5 |
| Brilliant Violet 650 anti-mouse CD69 | BioLegend | 104541 | 1:20 |
| Alexa Fluor 700 anti-mouse CD62L | BioLegend | 104426 | 1:40 |
| APC anti-mouse iNKT Tetramer | NIH Tetramer | PBS57-CD1d | 1:5 |
| | Core | | |
| PE anti-mouse iNKT Tetramer | NIH Tetramer | PBS57-CD1d | 1:5 |
| | Core | | |
| PE/Cyanine7 anti-mouse IFN-γ | BioLegend | 505825 | 1:5 |
| APC anti-mouse TNF-α | BioLegend | 506308 | 1:5 |

### Animals, Diet, and Antibiotic Exposure

Four-week-old female specific pathogen-free C57BL/6J mice (Jackson stock number: 000664) were purchased from The Jackson Laboratory. Mice were randomized into 5 mice per cage. All animals were housed in a temperature-controlled facility with a 12-hour light/dark cycle and ad libitum access to purified defined diets as follows. Low-fat diet (LFD) with 10% kcal from fat (D12450Ji Research Diets Inc.) was introduced 24 hours after arrival at the animal facility and mice remained on this diet for the duration of the study. LFD was chosen because donors were fed high fat diet (HFD) with 60% kcal from fat (D12492i Research Diets Inc.), and LFD is matched in micronutrients to the HFD, providing the optimal environment for FMT engraftment. Mice were housed for two weeks prior to intervention and bedding was mixed between cages weekly to normalize baseline microbiomes across cages to reduce cage effects. After a 2-week acclimation period, mice were treated with an antibiotic (ABX) cocktail (vancomycin, 0.5 g/L, neomycin, 1 g/L, ampicillin, 1 g/L, and cefoperazone, 1 g/L) in autoclaved water or plain autoclaved water in water bottles for eight days to ablate the commensal gut microbiome. Water intake was measured by water bottle weight daily. Body weight was measured weekly until the study endpoint.

### Fecal Microbial Transplant

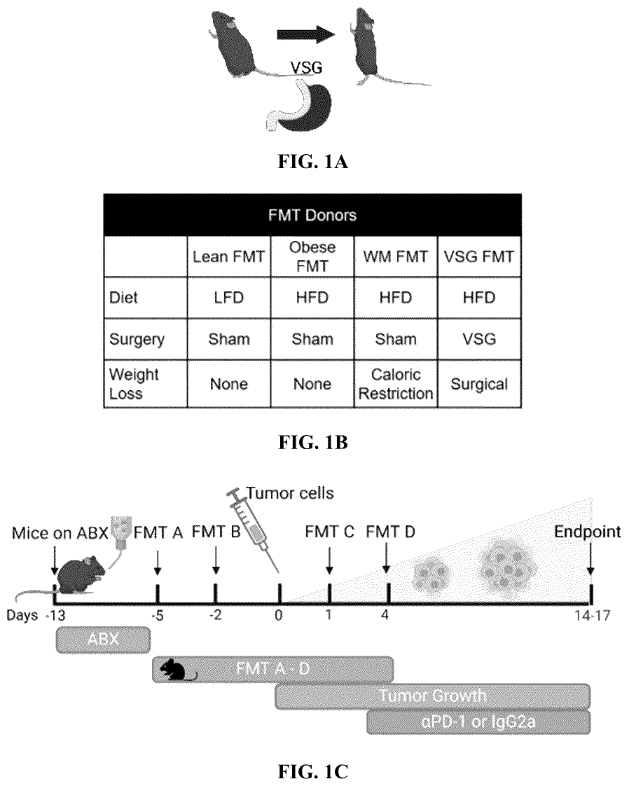

Fecal microbiota for FMT were collected from donors as part of a previously published study which reported impacts of obesity and bariatric surgery-mediated weight loss in BC (FIG. 1A, FIG. 1B, and FIG. 1C, Sipe L M, et al. Response to immune checkpoint blockade improved in pre-clinical model of breast cancer after bariatric surgery. Elife. 2022; 11: e79143). That study used specific pathogen free C57BL/6J females that were group housed and were fed either LFD to remain lean or made obese by HFD exposure for 16 weeks. In that study, all mice received sham surgery except one obese group that received VSG to induce weight loss with surgeries conducted as previously published and all mice remained on HFD.

In brief, surgeries were performed under isoflurane anesthesia. In VSG group, the stomach was clamped and 80% of the stomach was removed. The remaining stomach was sutured to create a tubular gastric sleeve. Obese and weight-matched (WM) caloric restriction control groups had a sham surgery wherein the abdominal laparotomy was performed, and light pressure was applied to the stomach with forceps. Mice were closed with sutures and received carprofen as an analgesic immediately prior to and for 3 days following surgery. Upon stable weight loss after VSG and caloric restriction or maintenance of obesity in sham controls, mice were injected orthotopically with E0771 BC cells to examine BC progression, which demonstrated that VSG led to reduced tumor progression compared to obese sham controls. At endpoint, cecal contents were collected for use in this study presented herein.

Fecal slurry was prepared from cecal contents from Sipe et al. mice (N=5-17 mice pooled per group). Cecal contents were resuspended at 20-30 mg/mL in glycerol/PBS with reduced oxygen under anaerobic conditions. The slurry was aliquoted for FMTs using a cut 200 μL tip and stored at −80° C. An aliquot from each group was used for quality control via 16S sequencing at University of Wisconsin Biotechnology Center DNA Sequencing Facility. Aliquots were thawed 1 hour prior to gavage on wet ice for FMTs.

To study the impact of transplanted microbiota on body weight, tumor progression, and response to immunotherapy, following ABX treatment, mice were assigned to one of six groups for the FMT controls or experimental groups (N=10 mice per group). There were two control groups: control (no ABX or gavage, ABX−/FMT−) and ABX only (no gavage, ABX+/FMT−). Four experimental groups included FMT gavage from the following donors: lean+sham (lean FMT), obese+sham (obese FMT), weight matched caloric restriction+sham (WM FMT) and VSG bariatric surgery (VSG FMT). The ABX cocktail was stopped 6 hours before the first gavage occurred and mice were given plain, sterile water in autoclaved water bottles. Mice were gavaged with 50 μL fecal slurry every three days for two weeks for a total of four FMT or control gavages as diagrammed in the study design (FIG. 1C). To ensure engraftment of microbiota from lean or obese donors accustomed to a specific diet (247), for 24 hours prior to and 24 hours following the first FMT, mice receiving microbes from donors that were previously on HFD were switched from LFD to HFD with 60% kcal from fat (D12492i Research Diets, Inc.) including obese FMT, WM FMT, and VSG FMT groups, while lean FMT recipients remained on LFD to match the donor diet. After the first FMT, all mice were maintained on LFD to minimize alterations to body weight. After the first two FMT gavages, mice were orthotopically injected with the E0771 BC cell line, then another two FMT gavages were subsequently administered (FIG. 1C).

### Human Fecal Collection and FMT Studies

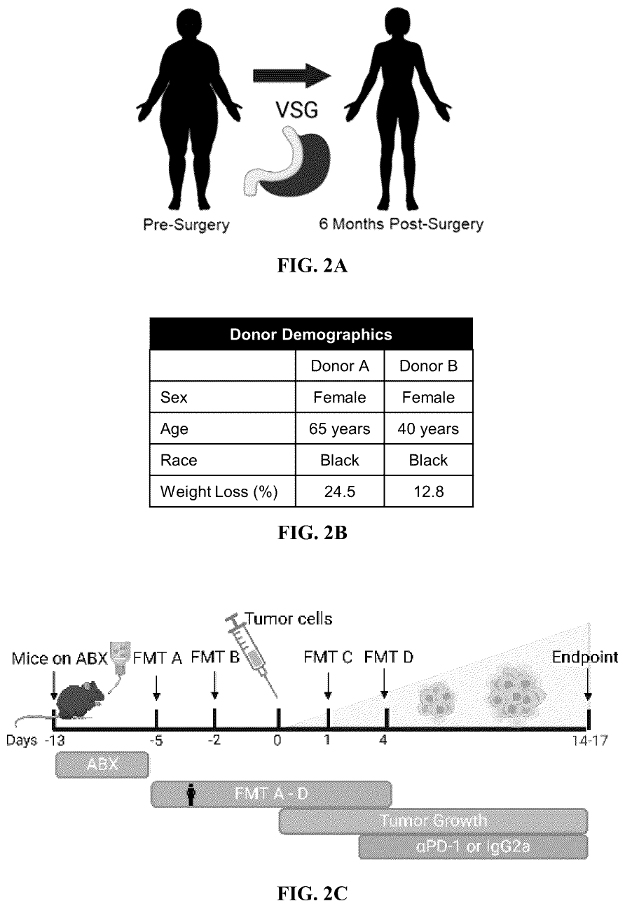

Fecal samples were collected from 65-year-old (Donor A) and 40-year-old (Donor B) African American female patients before bariatric surgery (pre-surgery) and 6 months after bariatric surgery (post-surgery) under IRB #20-07606-XP and IRB #22-08722-FB, respectively (FIG. 2A). The patients lost 24% and 13%, respectively, of starting body weight by the post-surgery collection timepoint (FIG. 2B) with pre-surgery BMI of 41.6 and 50.3, respectively, and post-surgery BMI of 31.4 and 44.6, respectively. Researchers were blinded to patient identities. Deidentified samples were resuspended at 25 mg/mL in glycerol/PBS with reduced oxygen under anaerobic conditions. Slurry was aliquoted using a cut 200 μL tip and stored at −80° C. An aliquot from each group was used for quality control via 16S sequencing. Aliquots were thawed 1 hour prior to gavage on wet ice for FMT, as described above.

For the human FMT studies, four-week-old specific pathogen free female C57BL/6J mice (purchased from The Jackson Laboratory as described above) were placed on Mediterranean or Western Diet (Research Diets Inc.) 24 hours after arriving at the animal facility. Mice were rested for two weeks and subjected to the same ABX cocktail and FMT study design as above for the mouse-to-mouse FMT experiment (FIG. 2C). After 8 days of ABX cocktail, mice received pre-surgery FMT gavage (N=20) or post-surgery FMT gavage (N=20). Following two gavages, mice were injected with E0771 cells as above, followed by two additional FMT gavages. ICB or IgG2a isotype control were administered as described below. Upon endpoint, tissues were collected as described below.

### Branched Chain Amino Acid Supplementation Study

Mice were subjected to BCAA supplementation to attempt to mimic the effects of the post-bariatric surgery microbiome. Five-week-old female specific pathogen-free C57BL/6J mice (The Jackson Laboratory) or BALB/cJ mice (Jackson stock number: 000651) were placed on LFD 24 hours after arriving at the animal facility. Mice were rested for two weeks prior to intervention and bedding was mixed between cages weekly to normalize baseline microbiomes across cages to reduce cage effects. After a 2-week acclimation period, mice were treated with a BCAA cocktail (leucine, 15 g/L, isoleucine, 15 g/L, and valine, 15 g/L) in autoclaved water while vehicle controls received plain autoclaved water in water bottles for the duration of the study. After 8 days of supplementation, mice were injected with E0771 cells as above or 4T1 cells. ICB or IgG2a isotype control were administered as described below. Water intake was measured by water bottle weight daily. Body weight was measured daily until the study endpoint. Upon endpoint, tissues were collected as described below.

### Cell Culture and Tumor Cell Implantation

E0771 BC cell line expressing luciferase was cultured. 4T1 cells were purchased from ATCC (CRL-2539). Cells tested negative for Mycoplasma (LT07-318 Lonza). Briefly, cells were cultured in RPMI 1640 containing 10% FBS, 2 mM GlutaMAX, 100 UI/mL penicillin, and 100 μg/mL streptomycin in a humidified chamber at 37° C. under 5% CO2. E0771 cells were orthotopically injected in the left fourth mammary fat pad of 8-week-old mice at 250,000 cells in 100 μL of 25% Matrigel Matrix High Concentration (354248 Corning), 75% RPMI with a final Matrigel concentration of 5 mg/mL. 4T1 cells were also orthotopically injected in the left fourth mammary fat pad of 8-week-old mice at 500,000 cells in 100 μL of 25% Matrigel Matrix High Concentration (354248 Corning), 75% RPMI with a final Matrigel concentration of 5 mg/mL. Starting seven days after implantation, tumor growth was monitored every third day by measuring the length and width of the tumor using digital calipers. Tumor volume was calculated using the following formula: Volume=(width) 2×(length)/2. No tumors failed to take in any of the studies. At experimental endpoint (days 13-20 after tumor cell injection), excised tumor mass was determined. The number of mice with regressed (undetectable) tumors compared to total palpable tumors at endpoint was recorded.

### Immune Checkpoint Blockade

Following BC cell injection, immune checkpoint blockade αPD-1 antibody (clone RMP1-14, BE0146 BioxCell) or rat IgG2a isotype control (clone 2A3, BE0089 BioxCell) were administered at 200 μg per mouse in 50 μL PBS by intraperitoneal (i.p.) injection. Antibody administration began 3 days after BC cell implantation. ICB or control were administered every 3 days until endpoint.

### Tissue and Blood Collection

At experimental endpoint, mice were fasted 4 hours prior to isoflurane anesthesia. Blood was collected via cardiac puncture into EDTA-coated syringes. Plasma was separated from other blood components by centrifugation at 1200×g for 45 min at 10° C. Mammary tumors, unaffected mammary fat pads from the contralateral side, gonadal adipose, mesenteric adipose (surrounding intestine), intestinal microbiota contents, spleen, liver, and brown adipose were weighed and either snap frozen in liquid nitrogen or digested into a single cell suspension for flow cytometry. All frozen samples were stored at −80° C. until analyzed.

### Microbiota Analysis

Cecal contents were collected from donors described and recipients at endpoint, flash-frozen, and stored at −80° C. for analysis. Fresh stool samples were collected prior to commencing the antibiotic cocktail to establish a baseline and upon completion of antibiotic regimen to determine to what extent antibiotics ablated the gut microbiota. Stool samples were collected on ice and stored at −80° C. for analysis.

For DNA isolation, previously developed methods were followed. Purified DNA samples underwent 16S library preparation and NextGen Illumina MiSeq sequencing at University of Wisconsin—Madison Bioinformatics Resources Core. Controls included empty tubes passed through an identical collection, extraction, and amplification process but remained free of DNA amplification. Paired-end reads were processed in R studio version 4.3.2 using the DADA2 pipeline to correct sequencing errors and to determine amplicon sequence variants (ASVs). ASVs were assigned taxonomy by comparison to Ribosomal Database Project release 18.0. Differential abundance of individual taxa was assessed using Corncob, while alpha diversity was calculated using vegan. Maaslin2 was used for correlation analyses.

### Metabolomic Analysis

Plasma collected at endpoint was analyzed for composition of circulating microbially derived metabolites including short-chain fatty acids, amino acids, and other metabolites. Compositions were measured at the University of Chicago Duchossois Family Institute Host-Microbe Metabolomics Facility. Metabolites were isolated via methanol extraction. Short chain fatty acid and amino acid groups were analyzed using GC-MS on an Agilent 7890A GC, Agilent 5975C MS detector with negative chemical ionization mode. Normalized peak areas were calculated by dividing raw peak areas of targeted analytes by averaged raw peak areas of internal standards. Correlation analyses were conducted using Maaslin2 in R studio version 4.3.2 or linear regression analysis in GraphPad Prism version 10.

### Flow Cytometric Analysis of Tumors

A portion of the excised tumors (~100 mg) were minced using scissors in RPMI media containing enzyme cocktail mix for mouse tumor dissociation (Miltenyi Biotec) following established protocols. Tumor pieces were further digested as per manufacturer's instructions, and digested tissue was filtered through 70 μm strainer to obtain a single cell suspension. Antibodies used (Table 1) were titrated and the separation index was calculated using FlowJo version 10 software for every study. NKT cells were labeled with soluble APC-tagged or PE-tagged PBS57-CD1d tetramers provided by the NIH Tetramer Core Facility to specifically identify iNKT subset (Table 1). In all studies, following red blood cell lysis (BioLegend), viability was determined by staining with Ghost dye (Tonbo Biosciences, Inc.) followed by FcR-blocking (Tonbo Biosciences, Inc.). Cells were stained with fluorescently labeled antibodies and fixed in Perm/fix buffer (Tonbo Biosciences Inc.). Stained cells were analyzed using a Bio-Rad ZE5 flow cytometer at the UTHSC Flow Cytometry and Cell Sorting Core. A minimum number of 1000 events were considered for analysis. Fluorescence minus one (FMO) stained cells and single color Ultracomp Beads (Invitrogen) were used as negative and positive controls, respectively. Data were analyzed using FlowJo version 10 software with FlowAI plug in cleanup. Gating schemata are shown for both myeloid and T cell-specific flow and NKT cell-specific staining.

For intracellular staining, single cell suspensions were stimulated with Cell Activation Cocktail (423303 Biolegend) for 3 hours to allow accumulation of intracellular cytokines prior to staining. Following cell surface marker staining, cells were fixed and permeabilized with Flow Cytometry Perm Buffer (Tonbo Biosciences, Inc.) overnight. Intracellular staining of IFNγ and TNFα was completed the following day.

### In Vitro BC Cell Branched Chain Amino Acid Assays

Cell proliferation of E0771 BC cells were detected using the IncuCyte S3 live-cell analysis instrument (Sartorius AG, Gottingen, DE, Germany). The cells were seeded into 96-well plates at a density of 1000 cells per well with 100 μL of complete medium. Following an overnight incubation for cell adhesion, a BCAA cocktail containing equal amounts of leucine, isoleucine, and valine was introduced in a dose-dependent manner at a concentration of 0 mM, 0.5 mM, 1 mM, and 2 mM alongside a positive chemotherapy control, Paclitaxel, at 10 nM concentration. Cell proliferation measurements were captured every 4 hours up to 72 hours post-treatment, assessing cell confluency normalized to the zero timepoint (%) as the primary parameter. Experiments are representative of N=6 technical replicates and N=3 biological replicates. Representative images are shown for vehicle, 2 mM BCAA, and Paclitaxel groups at 0 and 72 hours post-treatment.

Cellular viability was assessed using a CyQUANT (3-(4,5-dimethylthiazol-2-yl-)-2,5-diphenyl-2H-tetrazolium bromide) MTT cell proliferation assay kit (V13154 Invitrogen) at 72 hours post-treatment. A total of 12 mM of MTT stock solution was prepared by adding sterile PBS and 10 μL of solution was added to each well. Plates were incubated at 37° C. for 4 hours. A total of 100 μL of SDS-HCl solution was then added to each well and the plates were incubated at 37° C. for an additional 4 hours. Absorbance readings were measured at 570 nm on a Cytation 5 cell imaging multimode reader (BioTek, Santa Clara, CA). Experiments are representative of N=3 biological replicates and include N=6 technical replicates.

### Ex Vivo NKT Cell Branched Chain Amino Acid Assays

iNKT cells were isolated and expanded in culture following published protocols (319, 320). In brief, lymphocytes were isolated from female C57BL/6J mouse livers via Percoll gradient centrifugation (320) and FACS-sorted using soluble APC-tagged PBS57-CD1d tetramers provided by the NIH Tetramer Core Facility. Sorting was performed using a Cytek Aurora cell sorter in the UTHSC Flow Cytometry and Cell Sorting Core. Sorting yielded populations that were more than 95% PBS57-CD1d Tetramer positive. Cells were cultured and following expansion, cells were treated with BCAAs (leucine, 10 mM, isoleucine, 10 mM, valine, 10 mM) for 24 hours (205). At endpoint, intracellular flow cytometry staining was conducted as described above.

### Statistics

All analyses and figure generation were performed in R studio version 4.3.2 or GraphPad Prism version 10 (GraphPad Software, Inc.). Statistical differences between experimental groups were determined using one-way or two-way ANOVA (as noted in figure legends) with Fisher's LSD test for individual comparisons. Outliers were identified and excluded based on the ROUT method with Q=1% in GraphPad Prism. For body weight, water intake, and tumor volume over time within animals, data was treated as repeated measures. All data are shown as mean±SEM. p values <0.05 were considered statistically significant. Sample size was determined by power analysis calculations and pilot experiments.

### Study Approval

Murine studies were performed with approval and in accordance with the guidelines of the Institutional Animal Care and Use Committee (IACUC protocol #24.0504.0) at the University of Tennessee Health Science Center. Work was conducted in compliance with the National Institutes of Health Guide for the Care and Use of Laboratory Animals. Human sample collection was conducted in accordance with the Declaration of Helsinki and approved by the Institutional Review Board of the University of Tennessee Health Science Center (IRB #20-07606-XP, approved Jun. 2, 2022 and IRB #22-08722-FB, approved Feb. 27, 2025). Written informed consent was obtained from all patients.

### Example 1

### Microbes Transplanted from Bariatric Surgery Donors Reduced Tumor Burden in Recipient Mice

To investigate the dependence of improved immunotherapy effectiveness after bariatric surgery on the gut microbiome, a preclinical FMT study was conducted as shown in the study schema (FIG. 1C) using various murine controls or cecal contents from formerly obese bariatric surgery donors (vertical sleeve gastrectomy (VSG) FMT), formerly obese caloric restriction weight matched donors (WM FMT), donors that remained obese after sham surgery (obese FMT), and lean donors that remained lean after sham surgery (lean FMT) (FIG. 1B).

Female C57BL/6J mice were purchased from The Jackson Laboratory at 4 weeks of age and placed on low fat diet (LFD) containing 10% kcal from fat to establish baseline weights. Prior to FMT, a cocktail of broad-spectrum ABX composed of vancomycin, neomycin, ampicillin, and cefoperazone was used to ablate the commensal gut microbiome of all mice while antibiotic-free control mice (ABX−/FMT−) remained on plain autoclaved water. Water intake and body weights were measured daily during the 8-day antibiotic intervention period. The antibiotic cocktail was well-tolerated and did not impact water intake when compared to control cages that did not receive antibiotics.

Following 8 days of antibiotic treatment, the mice were transferred back to plain autoclaved water to cease antibiotic exposure. After a 6-hour washout period on plain water, the first FMT dose of donor cecal contents was administered by oral gavage, with control groups receiving no gavage. FMTs were administered every 3 days for a total of 4 FMT doses. E0771 BC cell injections occurred following the first 2 FMTs (FIG. 1C). Control mice included mice without antibiotic exposure or FMT gavages (ABX−/FMT−), mice receiving ABX but not FMT (ABX+/FMT−), mice receiving microbes from lean donors (lean FMT), and mice receiving microbes from caloric restriction weight matched donors (WM FMT).

FMTs and cancer progression did not impact body weight. At endpoint, multiple tissues relevant to obesity, tumor, or immunobiology were isolated including adipose tissue (fat depots) such as mesenteric, brown, mammary gland, and gonadal. No changes in adipose weights or spleen and liver mass were observed amongst any group.

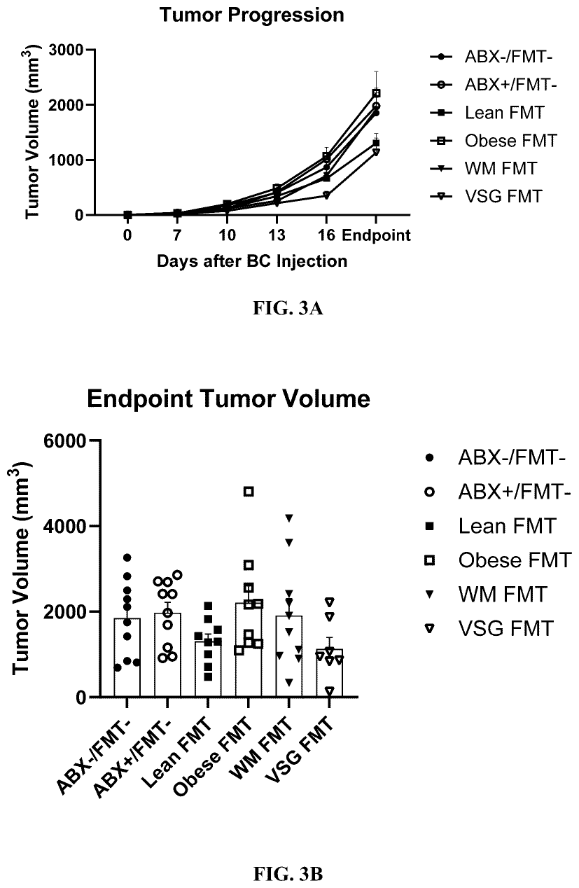

Tumor progression was quantified for 2.5 weeks prior to endpoint (FIG. 3A). Control mice without antibiotic exposure and in the absence of FMT gavages (ABX−/FMT−) or mice receiving just ABX but not FMT (ABX+/FMT−) displayed identical tumor progression (FIG. 3A, FIG. 3B) similar to previous findings. Lean FMT recipients had moderately slowed tumor progression when compared to controls (FIG. 3A, FIG. 3B), while mice receiving microbes from donors that lost weight via caloric restriction had no change in tumor progression compared to obese FMT (FIG. 3A, FIG. 3B). VSG FMT recipients did not significantly reduce tumor progression compared to obese FMT recipients in FMT alone (FIG. 3A, FIG. 3B). However, a modest reduction in tumor burden was observed in VSG FMT recipients (p=0.0517).

### Example 2

### Microbes Transplanted from VSG Donors Increased Immunotherapy Effectiveness in Recipient Mice

A subset of mice were subjected to the same FMT study design in Sipe et al. with the addition of αPD-1 ICB treatment or IgG2a isotype control. ICB was injected intraperitoneally every three days, starting when tumors first became palpable three days after injection (FIG. 1C). Control mice included mice without antibiotic exposure or FMT gavages (ABX−/FMT−) receiving either αPD-1 ICB or IgG2a isotype control. Water intake and body weights were not impacted by study design. While mesenteric and brown adipose did not demonstrate a change in mass after FMT or ICB, mammary gland and gonadal adipose demonstrated significantly increased weights in VSG recipients only after αPD-1 immunotherapy. Spleen and liver mass was not altered by FMT or ICB.

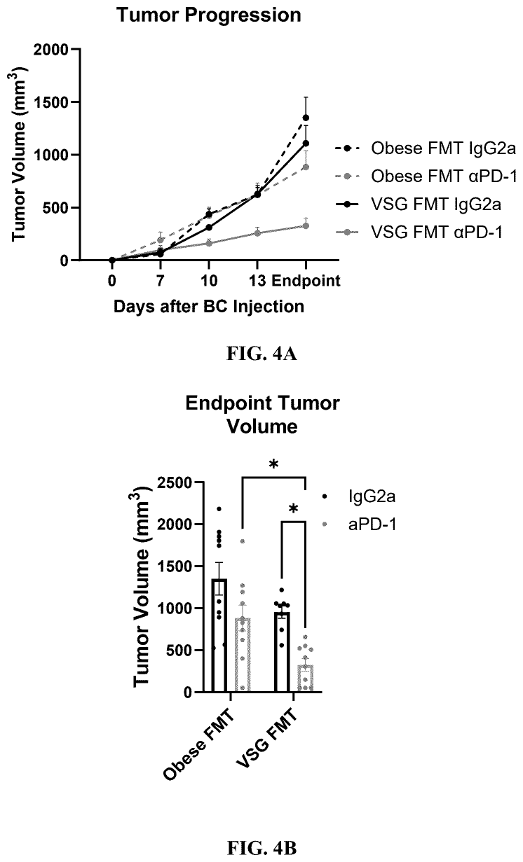

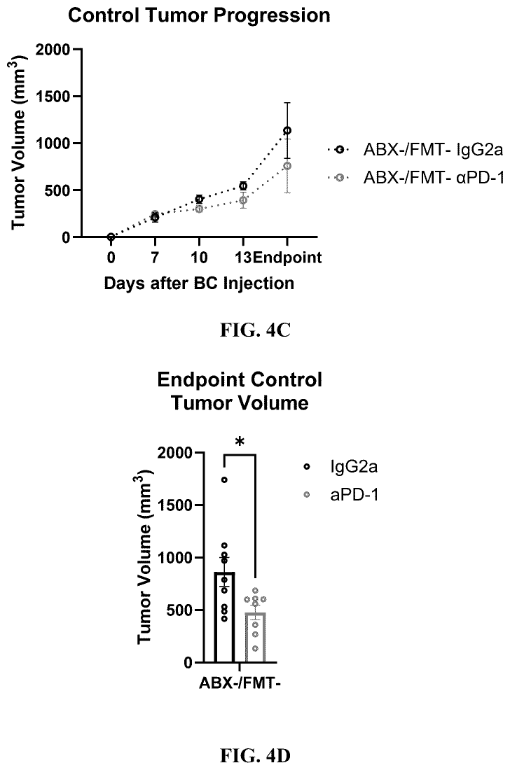

VSG FMT recipients did not significantly reduce tumor progression compared to obese FMT recipients in IgG2a controls (FIG. 4A, FIG. 4B, FIG. 4C, FIG. 4D). With αPD-1 ICB intervention, mice receiving obese FMT displayed only a moderate and insignificant reduction in tumor progression and endpoint volumes compared to IgG2a controls (FIG. 4A, FIG. 4B). Strikingly, VSG FMT recipients displayed a potently improved response to αPD-1 ICB, with significantly reduced tumor progression observed as early as day 10 post-cell injection (FIG. 4A, FIG. 4B).

Endpoint tumor volumes showed a substantial 71% reduction in volume after ICB in the VSG FMT recipients compared to a 35% reduction in obese FMT recipient controls, a 2-fold improvement in αPD-1 effectiveness comparing response of obese versus VSG donor FMTs (FIG. 4B). Control mice without antibiotic exposure and in the absence of FMT gavages (ABX−/FMT−) displayed only a moderate response to αPD-1 ICB, comparable to that observed in the obese FMT recipients (FIG. 4C, FIG. 4D). These results demonstrate an impact of microbes on immunotherapy efficacy unique to mice receiving microbes from donors after VSG.

### Example 3

### Reduced Tumor Progression after Bariatric Surgery is Evident after Transplant of Patient Pre- and Post-Surgery Stool

To demonstrate the translational relevance of the microbiome in BC progression and response to immunotherapy, patient fecal samples were collected prior to bariatric surgery (pre-surgery timepoint) and 6 months after bariatric surgery (post-surgery timepoint) following sustained weight loss of 25% and 13%, respectively (FIG. 2B). FMT was performed as described in FIG. 1A but used patient fecal samples instead of murine fecal samples (FIG. 2C). Also, for the patient FMT study, female C57BL/6J mice were placed on a Mediterranean or Western diet to provide the optimal environment for human microbiome engraftment.

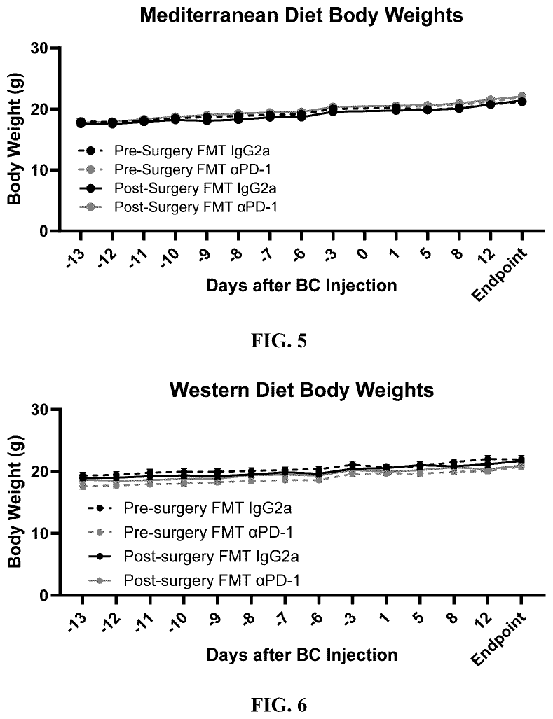

As above, ABX did not impact water intake (FIG. 5, FIG. 6). FMTs did not impact body weight, adipose tissue weights, or organ (spleen and liver) weights of recipient mice as mice remained lean throughout the study on both Mediterranean or Western diets. ICB treatment resulted in a minor but significant increase in mesenteric adipose weight in mice on Mediterranean diet.

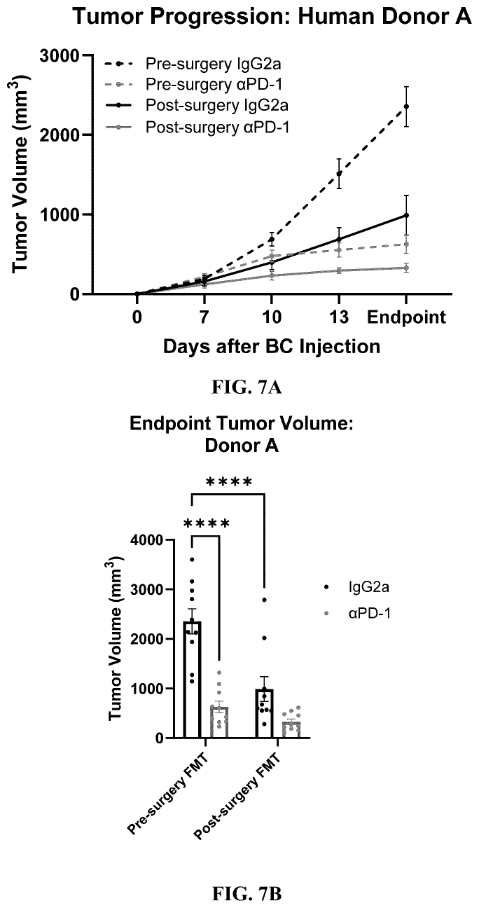

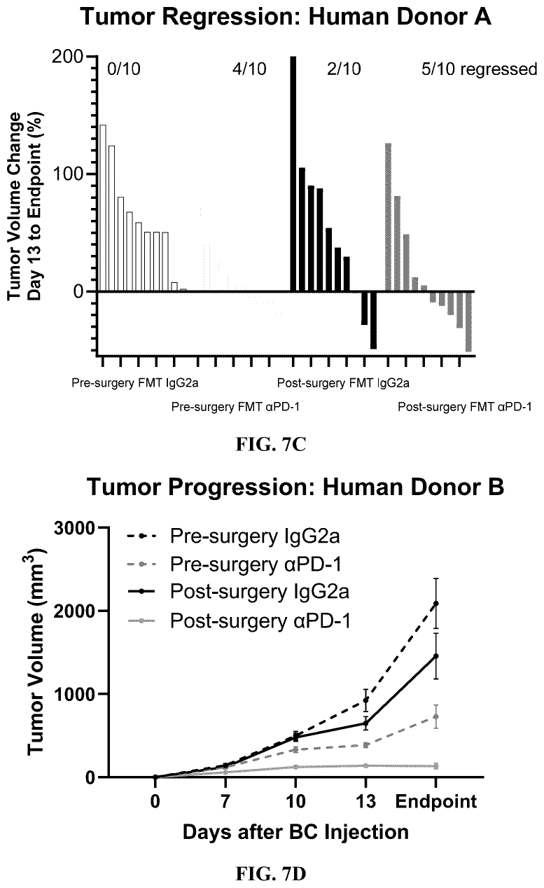

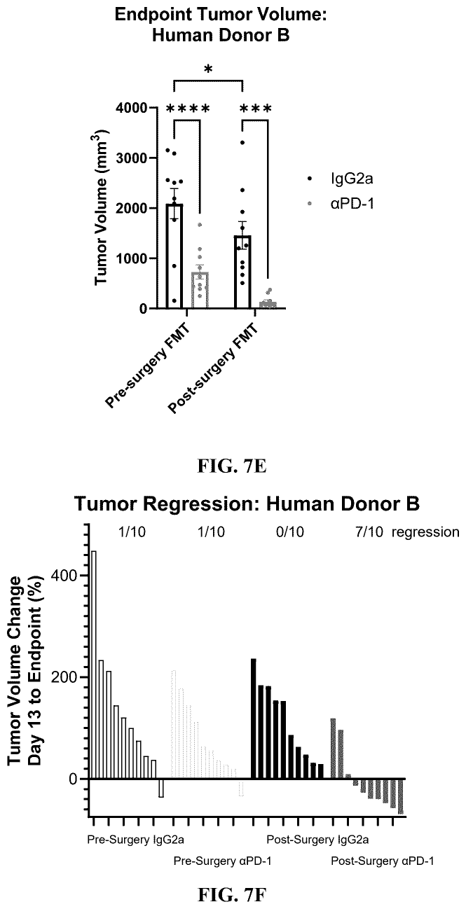

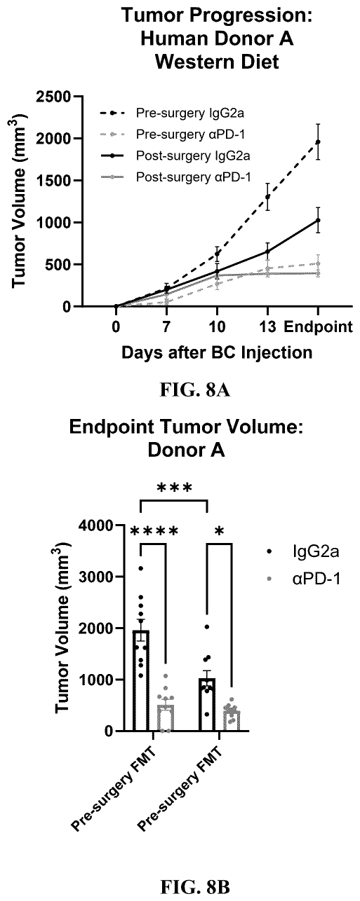

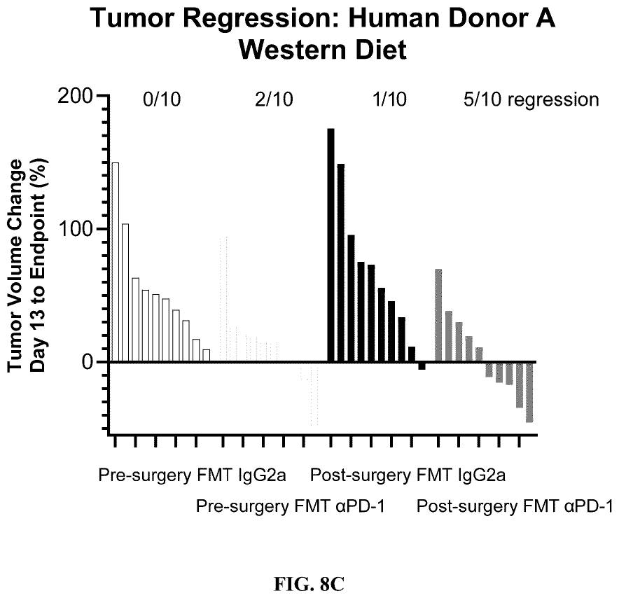

Analysis of tumor volumes over time demonstrated that mice receiving patient fecal content after bariatric surgery (post-surgery FMT) displayed significantly reduced tumor progression compared to pre-surgery donor content transplanted in IgG2a controls (FIG. 7A, FIG. 7B). Tumor endpoint volumes were significantly reduced by more than half in the mice receiving post-surgery FMT compared to mice receiving pre-surgery FMT from Donor A in the IgG2a controls (FIG. 7A, FIG. 7B) regardless of diet used. Indeed, mice receiving both Mediterranean (FIG. 7A, FIG. 7B) and Western diets (FIG. 8A, FIG. 8B) displayed a remarkably similar reduction in tumor burden following FMT with stool from Donor A. The impact of bariatric surgery alone comparing pre- and post-surgery FMT from Donor B was also significant with a 25% reduction in tumor volume in the IgG2a groups (FIG. 7D, FIG. 7E, FIG. 7F).

To determine if findings from the mouse-to-mouse FMT (FIG. 3A, FIG. 3B) translated to patients, the effectiveness of ICB treatment in both Donor A and Donor B FMT studies was tested. In mice receiving αPD-1, there was a significant anti-tumor response demonstrating reduced progression in pre-surgery FMT recipients in both Mediterranean and Western diets (FIG. 7A, FIG. 7B, FIG. 8A, FIG. 8B). The combination of ICB plus post-surgery microbiota demonstrated potently blunted tumor progression and reduced endpoint volumes, with significant reduction in Donor B (FIG. 7A, FIG. 7B, FIG. 8A, FIG. 8B).

The most remarkable response in the combination therapy using post-bariatric surgery FMT plus ICB was the regression of tumors that were palpable but shrunk in response to therapy. Data is shown as percent change in tumor volume from Day 13 to endpoint, and tumors are considered regressed if the percent change in volume is negative. In Donor A on Mediterranean diet, 50% of the tumors regressed (5/10 tumors) with an additional 2 minimally progressing below 15% (FIG. 7C). In Donor B, the combination therapy was even more potent with 70% (7/10 tumors) demonstrating regression, with an additional tumor minimally progressing. Additionally, this finding was not diet-dependent as observed in Donor A on Western diet, where 50% of the tumors regressed (5/10 tumors) from Day 13 of tumor growth to endpoint (FIG. 8C).

Tumor progression of the groups graphed separately for each donor and group highlight the progression of individual tumors throughout the course of the experiment and the effects of the combination FMT and ICB therapy. Thus, mice receiving microbes from patients post-bariatric surgery weight loss demonstrated significantly reduced tumor progression regardless of recipient diet, indicating that the gut microbiome associated with bariatric surgery after transplantation per se is effective in reducing tumor burden and inducing tumor regression. Furthermore, the combination therapy of post-bariatric surgery microbiome plus αPD-1 immunotherapy treatment led to robust tumor regression compared to pre-surgery recipients even with ICB therapy.

### Example 4

### Microbes from VSG and Obese Donors were Successfully Transplanted into Recipient Mice

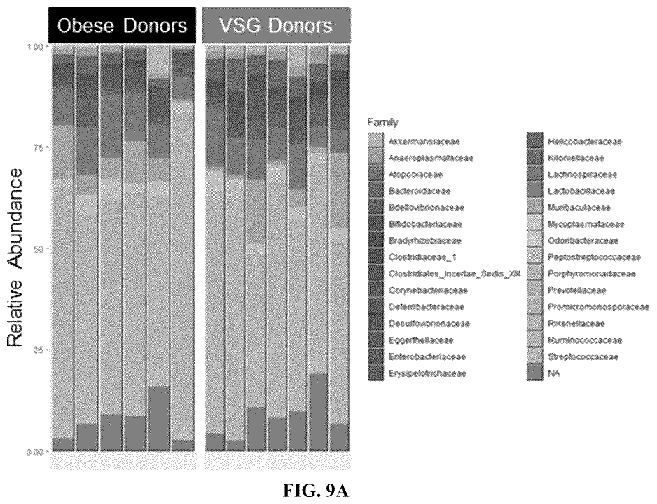

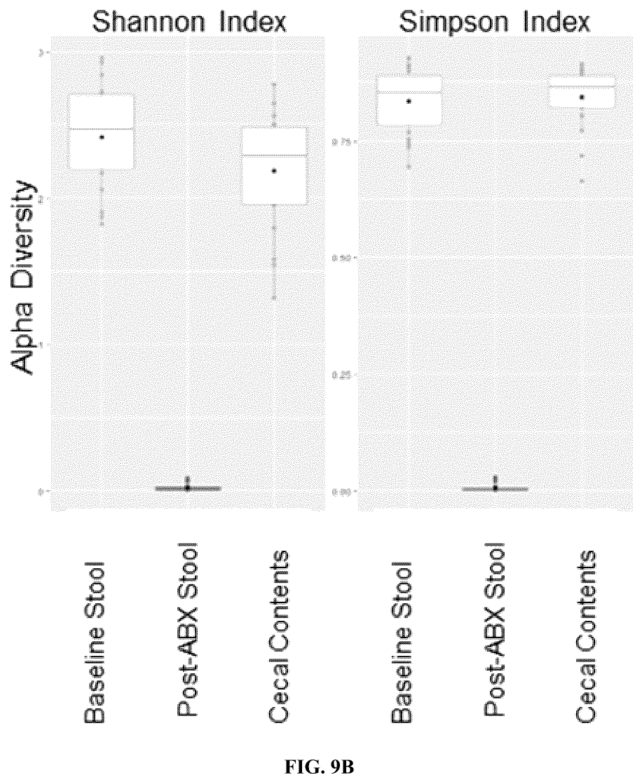

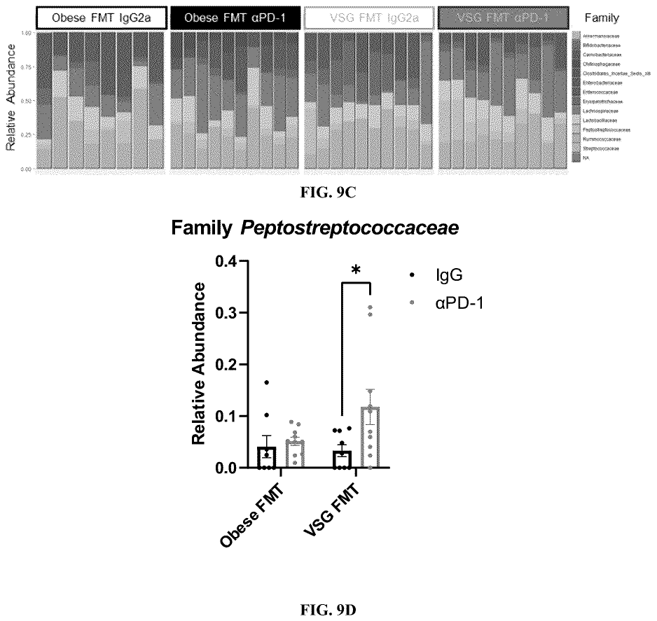

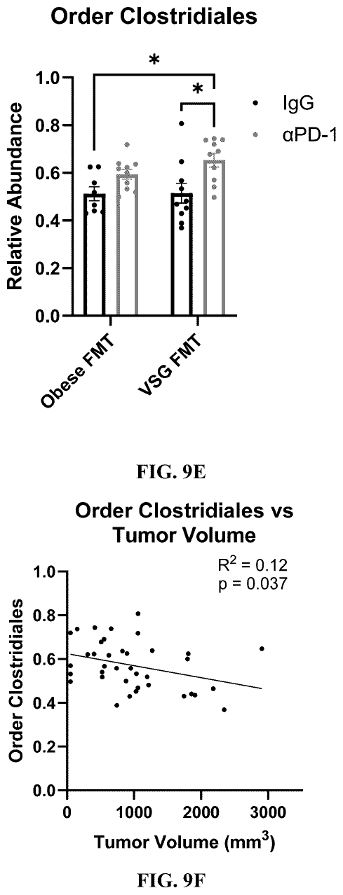

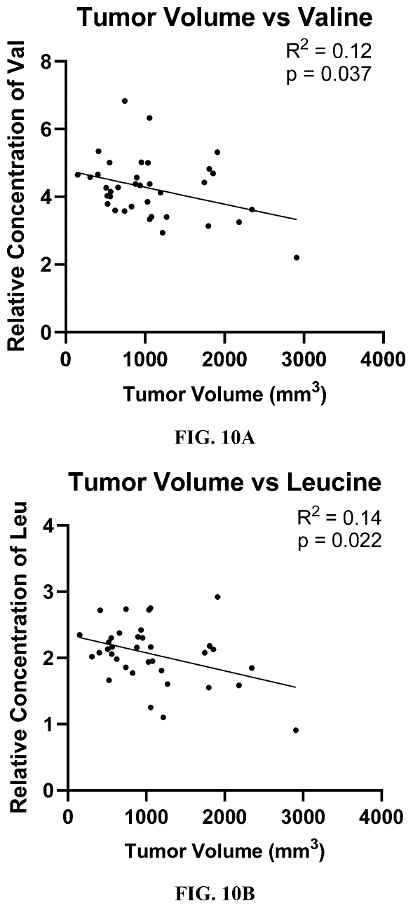

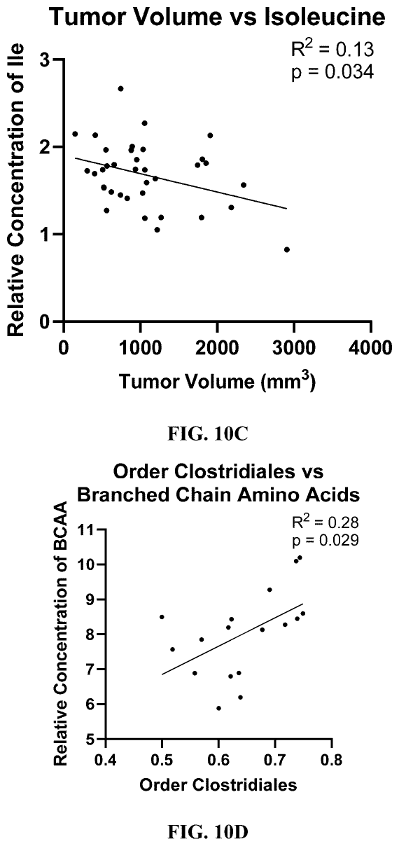

To confirm the efficacy of both the antibiotic cocktail and FMT experimental strategy, stool samples and endpoint cecal contents were collected for 16S microbiome sequencing from FMT donors as well as recipients from the mouse-to-mouse FMT study. Comparison of family level microbial abundance of endpoint cecal contents from the obese or VSG donor mice displayed minor variability in the microbiome with the greatest increase detected in Family Muribaculaceae (FIG. 9A).

Prior to the introduction of antibiotics or any other intervention (“Baseline Stool”), recipient mice displayed diverse gut microbiomes with high alpha diversity by both Shannon and Simpson indices (FIG. 9B). Following eight days of antibiotic treatment, stool samples were collected (“Post-ABX Stool”) and 16S sequencing demonstrated that the ABX cocktail was successful in ablating the commensal gut microbiome, as shown by dramatically reduced alpha diversity of Post-ABX Stool samples (FIG. 9B). Finally, recipient cecal contents harvested after FMT at study endpoint displayed regained alpha diversity metrics, indicating the successful engraftment of donor microbes (FIG. 9B). Taken together, these results support success of the present FMT design.

To examine impacts of FMT and ICB on obese versus VSG microbiome recipients, endpoint cecal contents in IgG2a controls compared to αPD-1 intervention were examined from mice examined in FIG. 6. Obese FMT compared to VSG FMT recipient contents revealed only moderate variation at the family level (FIG. 9C). Interestingly, αPD-1 ICB therapy induced unique alterations to the microbiome in obese FMT recipients that were absent in VSG FMT αPD-1 recipients as well as IgG2a controls of either donor group (FIG. 9C). In the obese FMT αPD-1-treated mice, increases were observed in Family Chitinophagaceae, Family Ruminococcaceae, and Genus Oscillibacter (FIG. 9C). In VSG FMT αPD-1 treated mice, Family Peptostreptococcaceae, a member of Order Clostridiales, was uniquely elevated amongst the 4 groups (FIG. 9D, FIG. 9E). Order Clostridiales was also uniquely elevated in VSG FMT αPD-1 treated mice (FIG. 9E), which significantly negatively associated with reduced tumor volume by MaAsLin2 correlation analysis (FIG. 9F). The gut microbiome, including members of Order Clostridiales, produces or modifies circulating metabolites, which prompted further experimentation to next conduct targeted metabolomic analysis on plasma samples.

### Example 5

### Elevated Circulating Branched Chain Amino Acid Concentrations Correlate with Improved Anti-Tumor Immunity

Order Clostridiales and other microbes influence circulating BCAA levels including leucine, isoleucine, and valine concentrations in the host. Because BCAAs are known to improve BC outcomes in pre-clinical models, the impacts of VSG and ICB on BCAA concentrations and anti-tumor immunity was determined. Metabolomic profiling by gas chromatography-mass spectrometry (GC-MS) of fasted plasma samples collected at study endpoint was conducted on microbially derived metabolites with 67 metabolites measured.

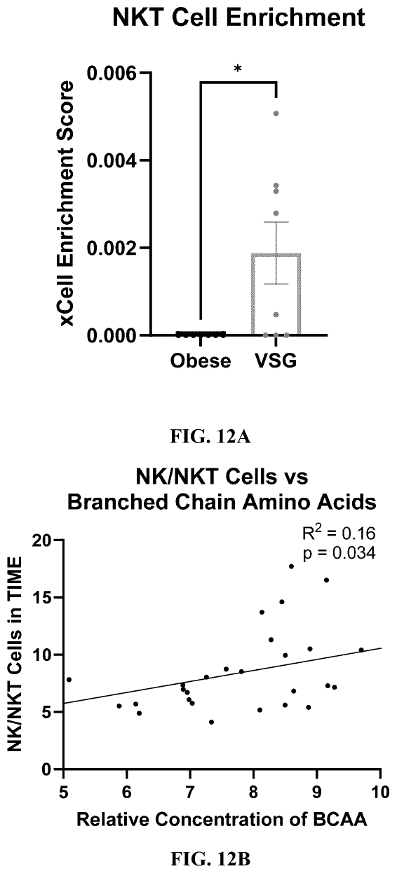

Linear regression analysis of individual BCAAs and tumor volume showed significantly negatively associated findings across all three BCAAs, with greater valine, leucine or isoleucine concentrations in smaller tumors and reductions in BCAA concentrations in larger tumors (FIG. 12A, FIG. 12B, FIG. 12C). Compared to αPD-1 treated obese FMT recipients, circulating BCAAs (the sum of leucine, isoleucine, and valine) was significantly greater in VSG FMT recipients after αPD-1 immunotherapy. Order Clostridiales relative abundance was found to be significantly positively correlated with BCAAs by MaAsLin2 analysis (FIG. 12D).

### Example 6

### Natural Killer T Cells were Uniquely Upregulated in VSG FMT Recipients after Anti-PD-1 Immunotherapy and Correlated with Elevated Circulating Branched Chain Amino Acids

BCAAs regulate the adaptive immune system, which could impact anti-tumor immunity. Flow cytometry was conducted on the tumors excised at endpoint to investigate the tumor immune microenvironment (TIME) using separate innate and adaptive panels. Interestingly, despite great differences in tumor progression (FIG. 4A), there were no significant changes in tumor immune cell content overall. Nor were there significant differences amongst the groups across many specific immune cells including M1-like MHCII high macrophage subtype, conventional dendritic cells (CDC) overall, cDC1 or cDC2 subtypes, CD4+ T cells and memory subsets, or CD8+ T cells and memory subsets between obese and VSG FMT or ICB recipients.

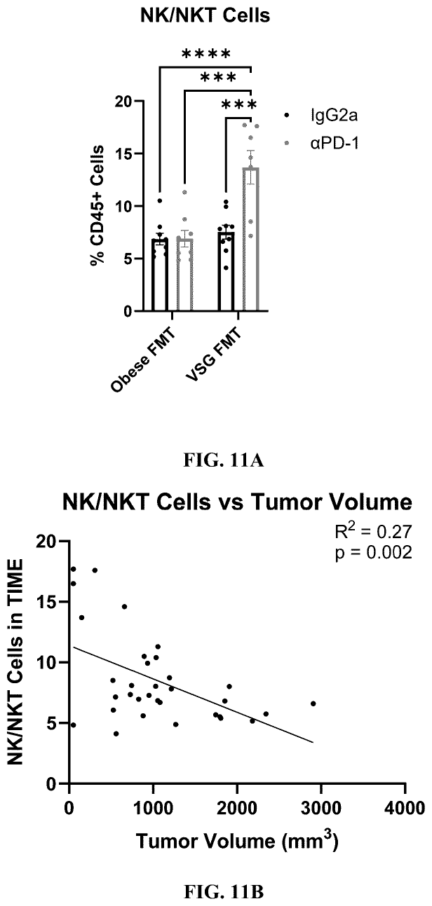

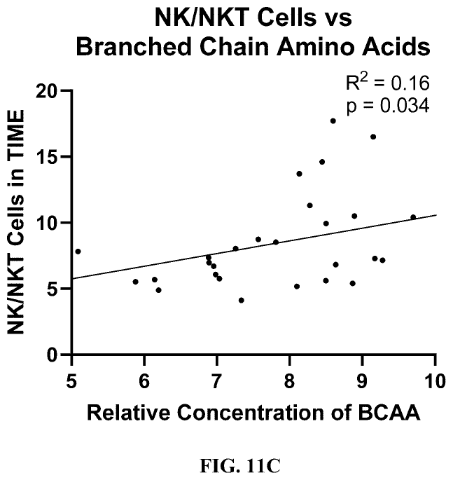

Surprisingly, natural killer (NK) and NKT cells, identified by the NK1.1 marker, were impacted in a manner that eclipsed other immune cell differences. NK1.1+NK/NKT cells were significantly and uniquely upregulated in VSG FMT recipients after αPD-1 immunotherapy (FIG. 11A), with an almost 2-fold increase in ICB treated mice compared to IgG2a controls detected only in the mice after receiving microbes from mice that had bariatric surgery. NK/NKT cells are effective in anti-tumor immunity; therefore, it follows that NK/NKT cell content significantly negatively correlated with endpoint tumor volume by linear regression analysis; the lower the NK/NKT cell content, the larger the tumors observed (FIG. 11B).

When immune cells were examined by linear regression analysis for correlations between circulating microbially derived metabolite concentrations and immune cell content within the TIME, BCAA concentrations significantly positively correlated with NK/NKT cell TIME content with 64 other metabolites not significantly correlated (FIG. 11A, FIG. 11B, FIG. 11C). There were no significant changes in circulating BCAA levels between obese or VSG donor mice. However, in donors from which the cecal content was isolated for FMT in FIG. 4A, NKT cell enrichment was significantly elevated in tumors from VSG donors when compared to obese donors (FIG. 12A). Importantly, NK cells were not enriched. Lastly, in the same donor mice, BCAA concentration correlated positively and significantly with NKT cell enrichment (FIG. 12B). Immunometabolic analysis of both donors and FMT recipients suggests a critical contribution of BCAAs and NK/NKT cells to improved response to immunotherapy in mice after VSG and, importantly, those receiving fecal content of donors after VSG which prompting a investigation into BCAA supplementation to determine direct impacts on response to ICB.

### Example 7

### Branched Chain Amino Acid Supplementation Mimics the Effects of the Post-Bariatric Surgery Microbiome to Boost Immunotherapy Effectiveness Through Elevations in Invariant Natural Killer T Cells

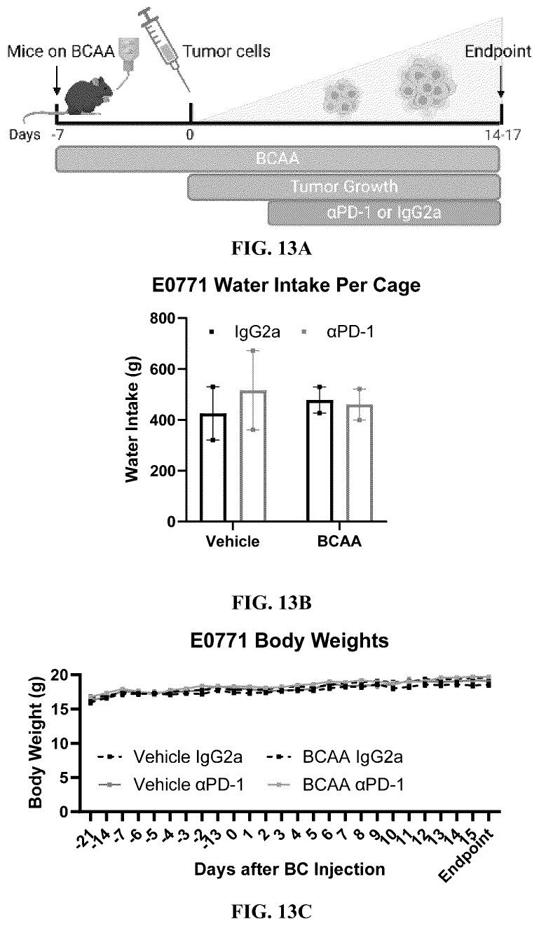

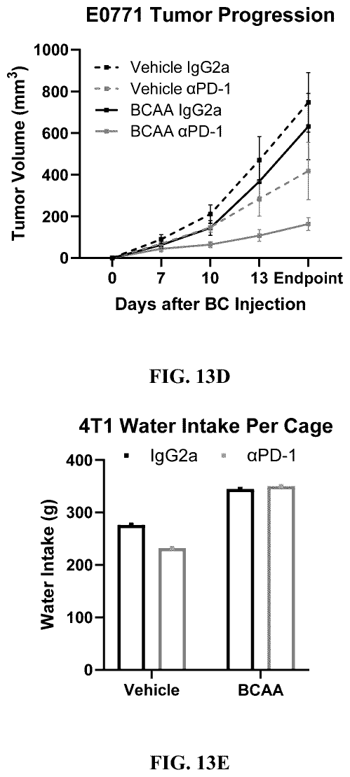

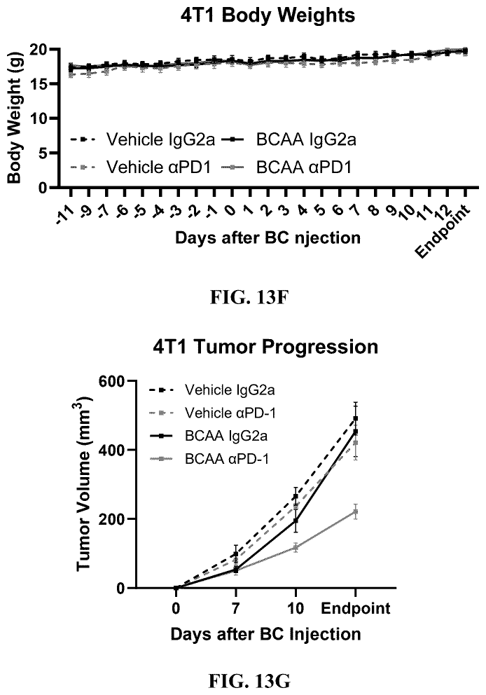

To directly test the relationship between circulating BCAA concentrations, NK/NKT cell content, and response to immunotherapy, two strains of mice were treated with oral BCAA supplementation to examine two different BC models. NKT cells can be divided into subsets based upon the structure of their TCR α-chain (invariant Type 1 and diverse Type II) and their cytokine production profile (T_{H}1-like iNKT1, T_{H}2-like iNKT2, Treg-like, and more). iNKT1 cells are classically immune activating, secreting cytokines such as IFNγ and TNFα, while iNKT2 cells are considered immunosuppressive, secreting cytokines such as IL-4 and IL-13. To confirm the subtype of NKT cells present in the TIME of BCAA supplemented mice, an α-galactosylceramide analog, PBS57, tetramer was used to specifically stain for iNKT cells. Female C57BL/6J or BALB/cJ mice were placed on LFD as in FIG. 1C and supplemented with autoclaved water containing equal concentrations of valine, leucine, and isoleucine or vehicle controls (FIG. 13A). E0771 or 4T1, respectively, orthotopic injections and immunotherapy were performed as described in FIG. 4-1A. As in FMT studies, water intake was monitored, and BCAA supplementation did not impact water intake, body weights, or mammary adipose weight compared to controls (FIG. 13B, FIG. 13C, FIG. 13D, FIG. 13E, FIG. 13F, FIG. 13G).

Measurement of tumor progression demonstrated that mice receiving BCAA supplementation displayed no change in tumor progression; however, in combination with ICB, both models demonstrated significantly improved responses to αPD-1 treatment (FIG. 13D, FIG. 13G). Mice receiving vehicle displayed a moderate 44.1% reduction in tumor progression after αPD-1, while mice receiving BCAA supplementation showed a significant 74.2% reduction in tumor volume after ICB in the E0771 model (FIG. 13D). Interestingly, the 4T1 model showed no response to ICB in the vehicle group demonstrating resistance to immunotherapy. However, a 51% reduction in tumor progression after ICB in the BCAA supplemented group was evident in the 4T1 model (FIG. 13G), indicting that ICB resistance has been overcome.

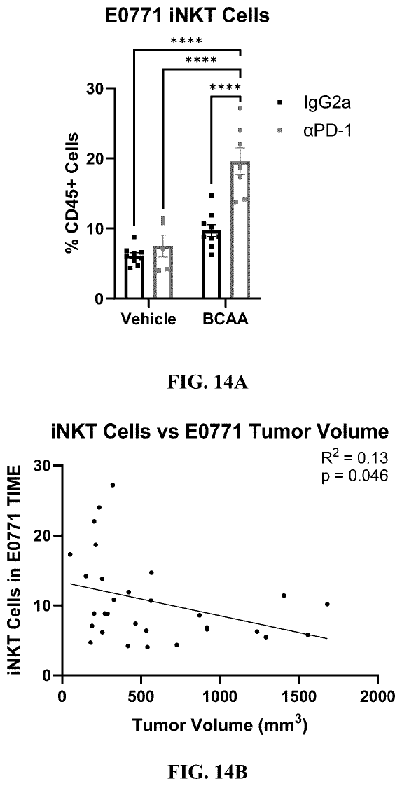

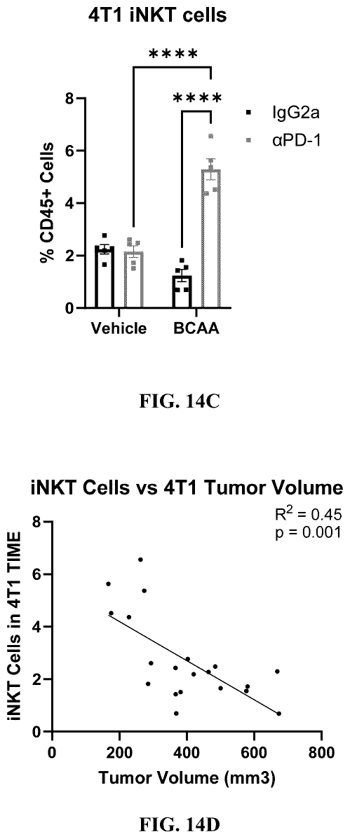

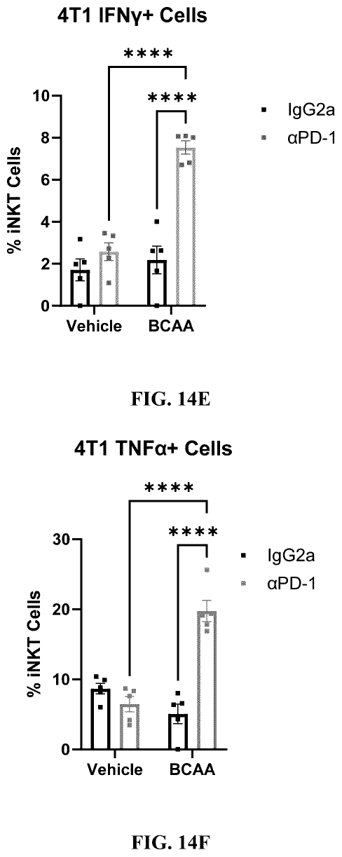

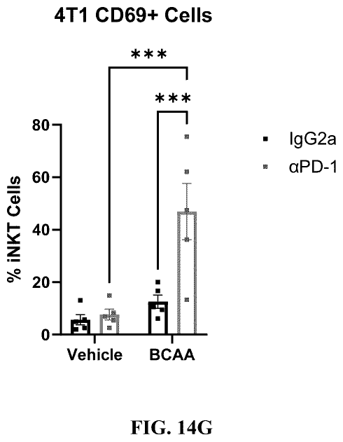

Similar to findings observed in the FMT studies, BCAA supplementation did not cause significant changes to the TIME in many innate or adaptive immune cell types as measured by flow cytometry. Because differences between NKT but not NK cells were evident in donor tumors with and without bariatric surgery, an α-galactosylceramide analog, PBS57, tetramer was used to specifically stain for the invariant NKT cell subtype and separate the NK and NKT cell populations present in the TIME. NK cell content was unchanged by BCAA supplementation; however, iNKT cell content was uniquely increased by 2-4-fold in mice receiving BCAA supplementation in combination with αPD-1 ICB in both the E0771 model (FIG. 14A) and the 4T1 model (FIG. 14C). iNKT cell TIME content correlated significantly negatively with tumor volume in both models (FIG. 14B, FIG. 14D). Thus, BCAA supplementation mimicked the effects of the post-bariatric surgery microbiome with combination therapy of BCAAs plus immunotherapy demonstrating potent anti-tumor impacts on BC in two pre-clinical models.

Importantly, BCAA supplementation not only resulted in increased iNKT cell content within the TIME but also elevated iNKT cell activation markers and proinflammatory cytokine production. CD69 expression was quantified as a marker of early iNKT cell activation. BCAA supplementation in combination with αPD-1 ICB resulted in increased activation marker CD69+iNKT cell staining 3.7-fold greater than IgG2a control therapy in the BCAA treated mice but not the vehicle controls (FIG. 14E).

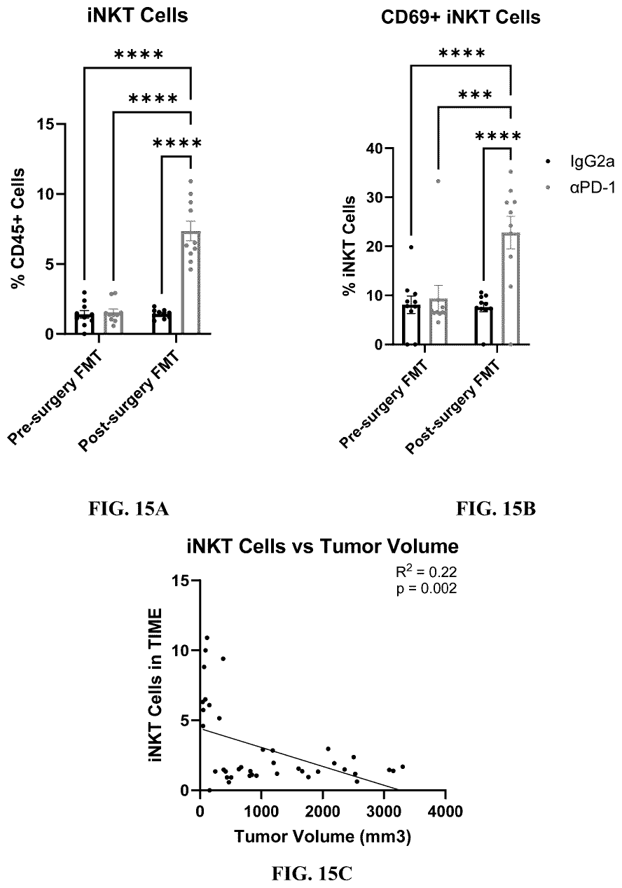

Flow cytometric analysis of the TIME from Donor B FMT (FIG. 7A) demonstrated increased iNKT cell content as well as elevated activation marker CD69+iNKT cell content in the tumors of mice receiving post-surgery FMT in combination with immunotherapy (FIG. 15A, FIG. 15B. iNKT cell content in the TIME significantly negatively correlated with tumor volume (FIG. 15C). No additional significant changes in other innate or adaptive immune cell types in the TIME were observed.

To determine the specific subtype of iNKT cells present in response to BCAA supplementation, intracellular flow cytometry staining revealed elevated content of both interferon gamma (IFNγ) and tumor necrosis factor alpha (TNFα) with BCAA treatment compared to controls (FIG. 14E, FIG. 14F). Secretion of these proinflammatory cytokines classifies the iNKT cells present in the TIME as belonging to the anti-tumor iNKT1 subtype. Taken together, oral BCAA supplementation replicated the effects of an FMT of post-bariatric surgery gut microbes on immune reprogramming to a more potent anti-tumor TIME.

BCAA supplementation potently improved αPD-1 ICB effectiveness with significant blunting of tumor progression and reduction in endpoint tumor burden. The improved tumor outcomes were accompanied by increased iNKT cell content within the TIME uniquely in BCAA supplementation recipients treated with αPD-1 ICB. NK cell content remained unchanged by BCAA supplementation and ICB treatment. Tetramer-positive iNKT cells correlated negatively with tumor volume at endpoint and demonstrated increased intracellular IFNγ and TNFα, indicating an improved anti-tumor immune response after BCAA supplementation.

### Example 8

### Branched Chain Amino Acid Treatment Increased iNKT Cell Production of Proinflammatory Cytokines In Vitro

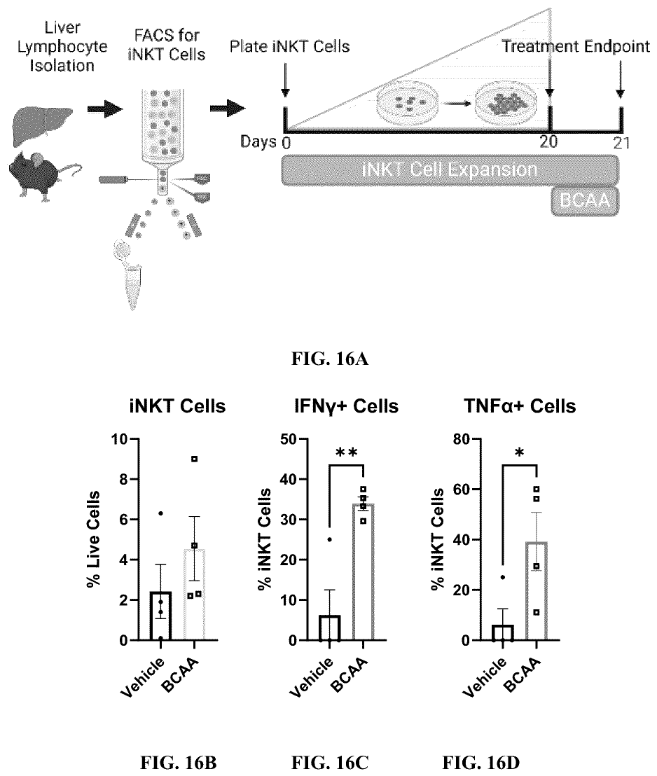

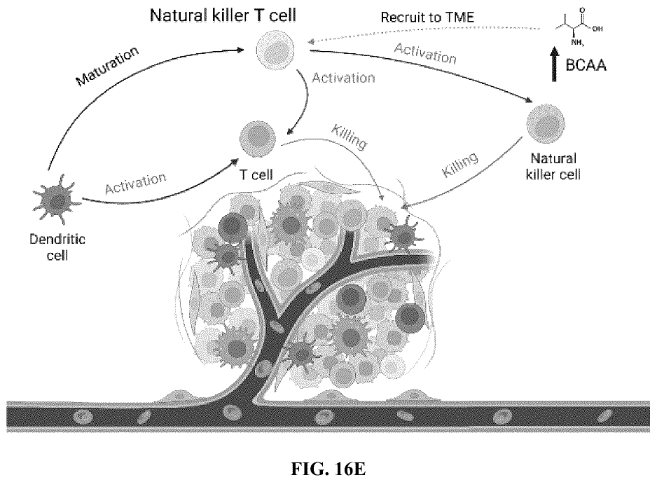

To investigate the direct activation of iNKT cells by BCAAs, iNKT cells were treated with BCAAs in vitro. Primary iNKT cells were isolated from female C57BL/6J mouse livers via fluorescence-activated cell sorting (FACS) (FIG. 16A). Ex vivo, cells were expanded in culture for 20 days. On day 20, expanded iNKT cells were treated with BCAAs for 24 hours prior to endpoint analysis by flow cytometry. BCAA treatment minimally but not significantly elevated iNKT cell proliferation (FIG. 16B). Within iNKT cells, greatly increased abundance of IFNγ and TNFα were detected in BCAA-treated cells relative to controls which essentially expressed no cytokines in 3 of the 4 replicates (FIG. 16C, FIG. 16D). In summary, these findings demonstrate that BCAAs play a direct role in iNKT cell cytokine production, which may underlie the potent anti-tumor immune response in mice supplemented with BCAA or those after bariatric surgery FMT.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The foregoing written specification and following examples are considered to be sufficient to enable one skilled in the art to practice the invention. Various modifications of the invention in addition to those shown and described herein will become apparent to those skilled in the art from the foregoing description and following examples and fall within the scope of the appended claims. The disclosures of all citations in the specification are expressly incorporated herein by reference.

To comply with written description and enablement requirements, all references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material. Incorporated by reference are the following references:

The various features and processes described above may be used independently of one another or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

All references cited herein are incorporated herein by reference in their entirety. To the extent publications and patents or patent applications incorporated by reference contradict the disclosure contained in the specification, the specification is intended to supersede and/or take precedence over any such contradictory material.

What is claimed is:

1. A method of treating breast cancer in a subject in need thereof, comprising:
administering to the subject an immune checkpoint inhibitor that blocks programmed cell death protein-1 (PD-1) or programmed death-ligand 1 (PD-L1); and
administering to the subject an effective amount of branched-chain amino acids (BCAAs) comprising leucine, isoleucine, and valine in a total daily dose sufficient to increase circulating BCAA plasma concentration by at least 10% relative to pre-treatment baseline,
wherein said BCAA administration activates invariant natural killer T (iNKT) cells within the tumor microenvironment as determined by an increase in iNKT1 cell frequency or activation marker expression relative to pre-treatment baseline, thereby improving therapeutic response to the immune checkpoint inhibitor.

2. The method of claim 1, wherein the BCAAs are administered orally as a nutritional supplement, pharmaceutical composition, or enteral formulation.

3. The method of claim 1, wherein the total daily dose is from about 0.1 g/kg/day to about 1.0 g/kg/day, determined based on achieving a target plasma BCAA concentration increase of at least 10% relative to pre-treatment baseline.

4. The method of claim 1, wherein leucine, isoleucine, and valine are administered in approximately equal amounts by weight at a leucine:isoleucine:valine ratio of 1:1:1, initiated at least seven days prior to initiation of immune checkpoint inhibitor therapy and continued for the duration of treatment.

5. The method of claim 1, wherein the iNKT1 cells activated within the tumor microenvironment are characterized by production of at least one of interferon-gamma (IFN-γ) or tumor necrosis factor-alpha (TNF-α), as determined by intracellular cytokine detection or equivalent immunological assay.

6. The method of claim 1, wherein the BCAAs are administered in combination with the immune checkpoint inhibitor to a subject whose breast cancer is resistant or refractory to immune checkpoint inhibitor monotherapy, wherein said BCAA administration sensitizes the tumor to immune checkpoint inhibitor therapy by increasing iNKT1 cell infiltration within the tumor microenvironment relative to pre-treatment baseline.

7. The method of claim 6, wherein the subject is a human patient with triple-negative breast cancer who has failed or is refractory to immune checkpoint inhibitor monotherapy.

8. The method of claim 1, further comprising measuring iNKT cell activation in the tumor microenvironment or peripheral blood of the subject by assessing at least one of iNKT cell frequency using CD1d tetramer staining or equivalent iNKT cell-specific detection reagent, expression of CD69 activation marker, or intracellular production of IFN-γ or TNF-α, and adjusting the BCAA dose based on an increase in iNKT cell activation relative to pre-treatment baseline.

9. The method of claim 8, wherein the method further comprises discontinuing or reducing BCAA administration when iNKT cell activation exceeds a predefined upper threshold indicative of potential immune-related adverse events.

10. The method of claim 1, further comprising measuring circulating BCAA plasma concentration in the subject prior to treatment initiation by a quantitative analytical method, wherein a circulating BCAA plasma concentration below a predefined threshold identifies the subject as a candidate for BCAA supplementation prior to or concurrent with immune checkpoint inhibitor administration.

11. A method of treating breast cancer in a subject in need thereof, comprising:
administering to the subject an immune checkpoint inhibitor that blocks programmed cell death protein-1 (PD-1) or programmed death-ligand 1 (PD-L1); and
administering to the subject a fecal microbiota transplantation (FMT) composition obtained from a post-bariatric surgery human donor who has undergone bariatric surgery at least three months prior to donation and achieved at least a 10% reduction in body weight relative to pre-surgical weight,
wherein the FMT composition is characterized by enrichment of order Clostridiales, including family Peptostreptococcaceae, relative to obese non-surgical controls, and wherein the FMT composition increases circulating branched-chain amino acid (BCAA) levels and invariant natural killer T (iNKT) cell activation within the tumor microenvironment of the subject relative to pre-treatment baseline,
thereby improving the therapeutic response of the subject to the immune checkpoint inhibitor.

12. The method of claim 11, wherein the subject undergoes gut microbiome ablation using a broad-spectrum antibiotic regimen comprising one or more of vancomycin, neomycin, ampicillin, and cefoperazone or equivalent broad-spectrum antibiotic regimen, administered for at least five days prior to administration of the FMT composition, and wherein the FMT composition is administered in at least two doses at intervals of no more than three days apart prior to initiation of immune checkpoint inhibitor therapy.

13. The method of claim 11, wherein the post-bariatric surgery human donor is selected based on a fecal microbiome profile demonstrating enrichment of order Clostridiales, including family Peptostreptococcaceae, relative to obese non-surgical controls, as determined by a nucleic acid-based microbial profiling method.

14. The method of claim 11, wherein the post-bariatric surgery human donor is selected based on stool metabolomic profiling demonstrating microbially derived BCAA levels above a predefined threshold relative to obese non-surgical controls, as determined by a quantitative analytical method.

15. The method of claim 11, wherein the improvement in therapeutic response comprises one or more of reduction in tumor volume of at least 50% relative to untreated controls, tumor regression to an impalpable state, or conversion of an immune checkpoint inhibitor-resistant tumor phenotype to an immune checkpoint inhibitor-responsive phenotype, as measured by caliper assessment, imaging modality, or equivalent tumor burden measurement.

16. A method of augmenting immune checkpoint blockade therapy in a subject with a solid tumor, comprising:
administering to the subject an immune checkpoint inhibitor that blocks programmed cell death protein-1 (PD-1) or programmed death-ligand 1 (PD-L1); and
administering to the subject an effective amount of branched-chain amino acids (BCAAs) comprising leucine, isoleucine, and valine sufficient to increase circulating BCAA plasma concentration by at least 10% relative to pre-treatment baseline, thereby activating iNKT1 cells within the tumor microenvironment and improving a therapeutic response to the immune checkpoint inhibitor,
wherein the solid tumor is breast cancer, triple-negative breast cancer, colorectal cancer, melanoma, or another solid tumor in which iNKT1 cell activation within the tumor microenvironment is associated with anti-tumor immune response.
